(19) United States
(12) Patent Application Publication
(10) Pub. No.: US 2025/0009908 A1
(43) Pub. Date: Jan. 9, 2025

(54) Fluorescently Labeled Immunoglobulin Single Variable Domains

(71) Applicant: Vrije Universiteit Brussel, Elsene (BE)

(72) Inventors: Sophie HERNOT, Elsene (BE); Pieterjan DEBIE, Elsene (BE)

(21) Appl. No.: 18/697,218

(22) PCT Filed: Oct. 5, 2022
(86) PCT No.: PCT/EP2022/077690

§ 371 Date: Mar. 29, 2024

(30) Foreign Application Priority Data
Oct. 5, 2021 (EP) 21200961.7

(51) Int. Cl. A61K 49/00; A61K 51/10

(52) U.S. Cl. CPC A61K 49/0032; A61K 49/0058; A61K 51/1093; A61K 2123/00

(57) ABSTRACT

The current invention relates to a conjugate comprising an immunoglobulin single variable domain conjugated to one or more detectable labels, wherein at least one of said labels comprises a fluorescent moiety, said fluorescent moiety having a structure chosen from formula (I) or formula (II). The current invention also relates to a conjugate comprising an immunoglobulin single variable domain conjugated to one or more detectable labels, wherein said conjugate exhibits less than 10 percent serum protein binding as measured by HPLC. The invention further relates to a pharmaceutical composition comprising aforementioned conjugate and the use of aforementioned conjugate or composition.

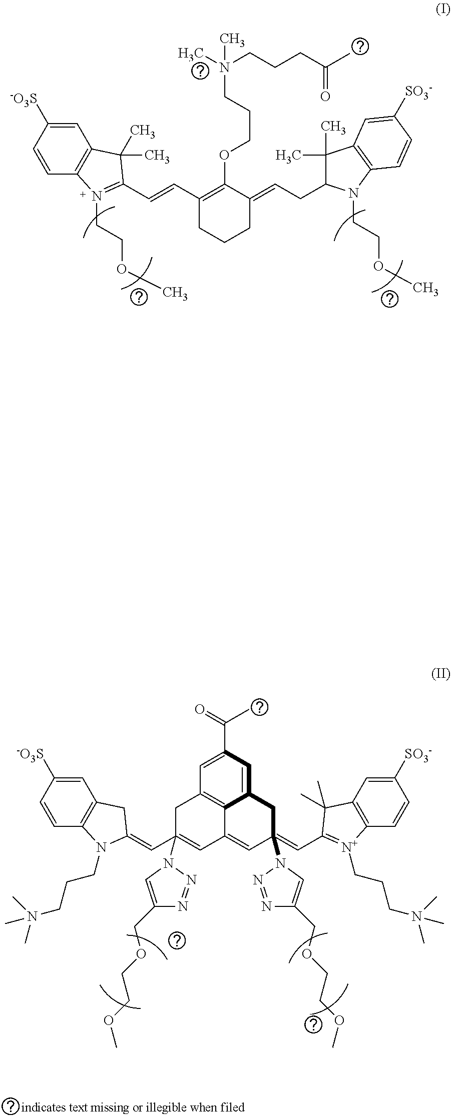

### FIELD OF THE INVENTION

The present invention pertains to the technical field of fluorescently labeled immunoglobulin single variable domains, a pharmaceutical composition comprising such fluorescently labeled immunoglobulin single variable domains and the use thereof.

### BACKGROUND

Fluorescence imaging is a relatively cheap, safe and high throughput in vivo imaging technology that relies on the sensitive detection of fluorescent signals emitted after excitation of a fluorescent contrast agent. Fluorescent imaging is commonly used in small animals to study specific biological processes at molecular and cellular level, also referred to as fluorescence molecular imaging. In clinical practice, fluorescence imaging is gaining significant attention as intraoperative guidance tool to aid the accuracy of surgical resections.

The specific accumulation of a fluorescent contrast agent in the tissue of interest, requires the stable conjugation of a fluorophore to a targeting molecule such as an antibody, antibody-fragment, protein scaffold, peptide or small molecule, recognizing a specific biomarker. While monoclonal antibodies bind their target with high affinity, their long biological half-life does entail certain disadvantages regarding specificity and the time necessary to attain sufficient contrast (typically several days). This can be overcome with smaller compounds exhibiting faster blood clearance.

As fluorophores, near-infrared (NIR) dyes are preferred because, in this region, light has the deepest tissue penetration and lowest scattering, and there is only minimal background autofluorescence. Several NIR dyes applicable for bioconjugation and in vivo imaging have been developed. IRDye800CW is a widely used dye for the design of targeted fluorescent tracers, but has also been reported to cause significant non-specific uptake of tracers in vivo. Alternatively, zwitterionic dyes with balanced surface charges in their structure have been shown to interact very little with serum proteins, resulting in less background signals as compared to IRDye800CW-labeled tracers. This has for instance been demonstrated for RGD-peptides labeled with ZW800-1 or FNIR-Tag-labeled antibodies. Unfortunately, so far it remains unpredictable what the impact of a chosen dye will be on the pharmacokinetic profile and targeting capabilities of a specific targeting moiety (or class of similar targeting agents) as the physicochemical characteristics of a dye will affect in an individual manner the overall molecular charge, charge distribution and hydrophobicity of a tracer, and thus its blood clearance, excretion pathway, specific and non-specific uptake, metabolization etc.

Hence in the field of in vivo medical imaging there is a need for fluorescently labeled targeting molecules showing improved pharmacokinetic properties.

### SUMMARY OF THE INVENTION

The present invention and embodiments thereof serve to provide a solution to one or more of the above-mentioned disadvantages. To this end, the present invention relates to a conjugate according to claim 1. Said conjugate comprises an immunoglobulin single variable domain conjugated to one or more detectable labels, wherein at least one of said labels comprises a fluorescent moiety, said fluorescent moiety having a structure chosen from formula I or formula II:

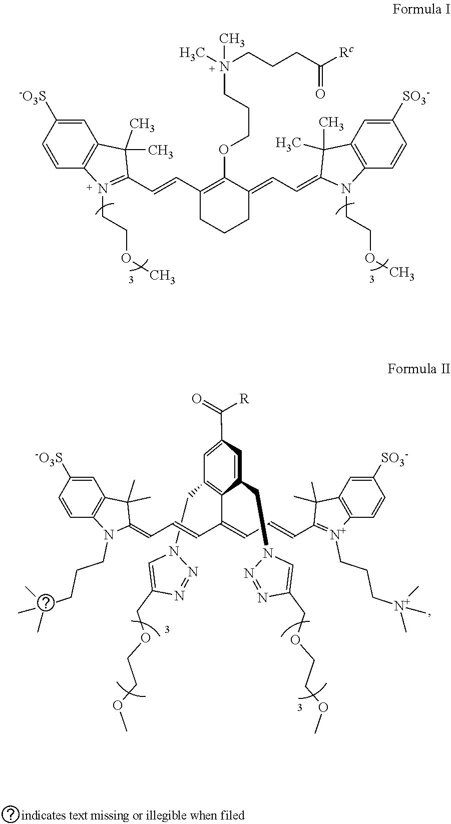

wherein R^{c} and R allow conjugation to the immunoglobulin single variable domain.

The inventors found that the conjugate according to claim 1 has an optimal biodistribution profile when administered in vivo, showing exclusive renal clearance, an ultralow imaging background and a high signal-to-background ratio as from early time points after injection.

In addition, the present invention also relates to a conjugate comprising an immunoglobulin single variable domain conjugated to one or more detectable labels according to claim 2. Said conjugate exhibits less than 10 percent serum protein binding as measured by HPLC. The binding of conjugates to serum proteins is an important process that determines the activity and pharmacokinetics of the conjugate in the body. After being distributed around the circulating system, conjugates bind to serum proteins in varying degrees. Because the conjugate according to claim 2 interacts very little with serum proteins, this will result in less background signals and a more desirable pharmacokinetic profile when the conjugate is used for in vivo medical imaging.

Preferred embodiments of the conjugates are shown in any of the claims 3 to 7.

In another aspect, the present invention relates to a pharmaceutical composition according to claim 8, comprising aforementioned conjugate.

In a further aspect, the present invention relates to a use according to claim 9, wherein aforementioned conjugate or composition is used for in vivo medical imaging.

Preferred embodiments of the use are shown in any of the claims 10 to 13.

### DESCRIPTION OF FIGURES

The following description of the figures of specific embodiments of the invention is merely exemplary in nature and is not intended to limit the present teachings, their application or uses. Throughout the drawings, corresponding reference numerals indicate like or corresponding parts and features.

The graphs in FIGS. 1-2 show data from example 1, more specifically, quality control by Size Exclusion Chromatography (panel A), excitation and emission spectra (panel B) and serum protein binding (panel C) of different fluorescently labeled conjugates, including conjugates according to an embodiment of the invention.

FIG. 3 shows data from example 1, more specifically, dorsal (A) and ventral (B) images acquired in HER2⁺ tumor-bearing mice at 1 h, 3 h, 6 h, 12 h, and 24 hours post-injection of different fluorescently labeled conjugates (all conjugated to the 2Rs15dNT-sdAb), including conjugates according to an embodiment of the invention. Tumor (T), kidneys (Kd), liver (L) and bladder (BI) are indicated. Exposure time and image scaling were chosen to display similar tumor signal for the different conjugates. FIG. 3C shows quantification of the tumor-to-muscle ratio in function of time for the different conjugates.

FIG. 4 shows data from example 1, more specifically, ex vivo analysis of organs and tissues at 1 or 24 h post-injection of different fluorescently labeled conjugates (all conjugated to the 2Rs15dNT-sdAb), including conjugates according to an embodiment of the invention. FIG. 4A shows representative fluorescent images of organs at 1 h post-injection. FIG. 4B shows quantification of the tumor-to-organ ratios.

FIG. 5 shows data from example 1, more specifically, dorsal (A) and ventral (B) images acquired in EGFR+tumor-bearing mice at 1 h, 3 h, 6 h, 12 h, and 24 hours post-injection of different fluorescently labeled conjugates (different fluorescent labels conjugated to the 7D12.6His-sdAb), including conjugates according to an embodiment of the invention. Tumor (T), kidneys (Kd), liver (L) and bladder (BI) are indicated. Exposure time and image scaling were chosen to display similar tumor signal for the different tracers. FIG. 5C shows quantification of tumor-to-contralateral muscle ratio in function of time for the different conjugates.

FIG. 6 shows data from example 1, more specifically, ex vivo fluorescent images of organs at 1 h post-injection of different fluorescently labeled conjugates (different fluorescent labels conjugated to the 7D12.6His-sdAb), including conjugates according to an embodiment of the invention. FIG. 6A shows representative fluorescent images of organs at 1 h post-injection. Exposure time and image scaling were chosen to display similar tumor signal for the different tracers. FIG. 6B shows quantification of the tumor-to-organ ratios at 1 h and 24 h post-injection.

FIG. 7 shows data from example 1, more specifically, representative in vivo dorsal and ventral fluorescence images acquired 1 h after injection of CEA5.6His-FNIR-Tag or CEA5.6His-s775z, which are both conjugates according to an embodiment of the invention (FIG. 7A), quantification of in vivo tumor-to-muscle ratio over time (FIG. 7B), fluorescence intensity measurements of excised organs and tissues, 1 h post-injection (FIG. 7C) and ex vivo tumor-to-organ ratios at 1 h (FIG. 7D).

FIG. 8 shows data from example 2, more specifically, representative in vivo dorsal and ventral fluorescence images acquired 1 h after injection of uPAR13.6His-s775z (FIG. 8A), fluorescence intensity measurements of excised organs and tissues, 1 h post-injection (FIG. 8B) and ex vivo tumor-to-organ ratios at 1 h postinjection (FIG. 8C).

FIG. 9 shows data from example 3, more specifically, representative in vivo dorsal and ventral fluorescence images acquired 1 h after injection of R3B23.6His-IRDye800CW (top panel) or R3B23.6His-s775z (bottom panel).

### DETAILED DESCRIPTION OF THE INVENTI ON

The present invention concerns conjugates comprising an immunoglobulin single variable domain conjugated to one or more detectable labels, compositions comprising said conjugate and uses of said conjugate or composition.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless 25 the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

“Immunoglobulin single variable domain” as used herein defines molecules wherein the antigen binding site is present on, and formed by, a single immunoglobulin domain (which is different from conventional immunoglobulins or their fragments, wherein typically two immunoglobulin variable domains interact to form an antigen binding site). It should however be clear that the term “immunoglobulin single variable domain” does comprise fragments of conventional immunoglobulins wherein the antigen binding site is formed by a single variable domain.

Generally, an immunoglobulin single variable domain will have an amino acid sequence comprising 4 framework regions (FR1 to FR4) and 3 complementarity determining regions (CDR1 to CDR3), preferably according to the following formula (1): FR1-CDR1-FR2-CDR2-FR3-CDR3-FR4 (1), or any suitable fragment thereof (which will then usually contain at least some of the amino acid residues that form at least one of the complementarity determining regions). Immunoglobulin single variable domains comprising 4 FRs and 3 CDRs are known to the person skilled in the art and have been described.

Typical, but non-limiting, examples of immunoglobulin single variable domains include light chain variable domain sequences (e.g. a VL domain sequence) or a suitable fragment thereof, or heavy chain variable domain sequences (e.g. a VH domain sequence or VHH domain sequence) or a suitable fragment thereof, as long as it is capable of forming a single antigen binding unit. Thus, according to a preferred embodiment, the immunoglobulin single variable domain is a light chain variable domain sequence (e.g. a VL domain sequence) or a heavy chain variable domain sequence (e.g. a VH domain sequence); more specifically, the immunoglobulin single variable domain is a heavy chain variable domain sequence that is derived from a conventional four-chain antibody or a heavy chain variable domain sequence that is derived from a heavy chain antibody. The immunoglobulin single variable domain may be a domain antibody (“dAB” or “dAb”), or a single domain antibody (“sdAB” or “sdAb”), or a, or a VHH domain sequence or another immunoglobulin single variable domain, or any suitable fragment of any one thereof. The immunoglobulin single variable domains, generally comprise a single amino acid chain that can be considered to comprise 4 “framework sequences” or FR's and 3 “complementary determining regions” or CDR's (as defined herein). It should be clear that framework regions of immunoglobulin single variable domains may also contribute to the binding of their antigens. The delineation of the CDR sequences (and thus also the FR sequences) can be based on the IMGT unique numbering system for V-domains and V-like domains. Alternatively, the delineation of the FR and CDR sequences can be done by using the Kabat numbering system as applied to VHH domains from Camelids.

It should be noted that the immunoglobulin single variable domains as binding domain moiety in their broadest sense are not limited to a specific biological source or to a specific method of preparation. The term “immunoglobulin single variable domain” encompasses variable domains of different origin, comprising mouse, rat, rabbit, donkey, human, shark, camelid variable domains. According to specific embodiments, the immunoglobulin single variable domains are derived from shark antibodies (the so-called immunoglobulin new antigen receptors or IgNARs), more specifically from naturally occurring heavy chain shark antibodies, devoid of light chains, and are known as VNAR domain sequences. Preferably, the immunoglobulin single variable domains are derived from camelid antibodies. More preferably, the immunoglobulin single variable domains are derived from naturally occurring heavy chain camelid antibodies, devoid of light chains, and are known as VHH domain sequences.

The term “VHH domain sequence” is, as used herein, is interchangeably with the term “single domain antibody fragment (sdAb)” and refers to a single domain antigen binding fragment. It particularly refers to a single variable domain derived from naturally occurring heavy chain antibodies and is known to the person skilled in the art. VHH domain sequences are usually derived from heavy chain only antibodies (devoid of light chains) seen in camelids and consequently are often referred to as

VHH antibody or VHH sequence. Camelids comprise old world camelids (Camelus bactrianus and Camelus dromedarius) and new world camelids (for example Lama paccos, Lama glama, Lama guanicoe and Lama vicugna). The small size and unique biophysical properties of VHH domain sequences excel conventional antibody fragments for the recognition of uncommon or hidden epitopes and for binding into cavities or active sites of protein targets. VHH domain sequences are stable, survive the gastro-intestinal system and can easily be manufactured. Therefore, VHH domain sequences can be used in many applications including drug discovery and therapy, but also as a versatile and valuable tool for purification, functional study and crystallization of proteins.

The VHH domain sequences of the invention generally comprise a single amino acid chain that can be considered to comprise 4 “framework regions” or FR's and 3 “complementarity determining regions” or CDR's, according to formula (1) (as defined above). The term “complementarity determining region” or “CDR” refers to variable regions in VHH domain sequences and contains the amino acid sequences capable of specifically binding to antigenic targets. These CDR regions account for the basic specificity of the VHH domain sequences for a particular antigenic determinant structure. Such regions are also referred to as “hypervariable regions.” The VHH domain sequences have 3 CDR regions, each non-contiguous with the others (termed CDR1, CDR2, CDR3). The delineation of the FR and CDR sequences is often based on the IMGT unique numbering system for V-domains and V-like domains. Alternatively, the delineation of the FR and CDR sequences can be done by using the Kabat numbering system as applied to VHH domains from Camelids. As will be known by the person skilled in the art, the VHH domain sequences can in particular be characterized by the presence of one or more Camelidae hallmark residues in one or more of the framework sequences (according to Kabat numbering).

As used herein, the term “cancer” or “tumor” refers to any neoplastic disorder, including prostate cancer, liver cancer, head and/or neck cancer (e.g. throat cancer, pharyngeal squamous cell carcinoma), brain cancer, skin cancer (e.g. melanoma), bladder cancer, ovarian cancer (e.g. ovarian carcinoma), uterus cancer, lung cancer, breast cancer (e.g. mammary adenocarcinoma), sarcoma, cervix cancer, vulva cancer, hematologic cancers (e.g. chronic leukemia, mastocytoma), renal cancer (e.g. renal cell cancer), bone cancer, and/or gastrointestinal cancers (e.g. colorectal cancer, pancreas cancer, gastric cancer, esophageal cancer).

“Radionuclide” as used herein refers to an atom that has excess nuclear energy, making it unstable. This excess energy can be used in one of three ways: emitted from the nucleus as gamma radiation; transferred to one of its electrons to release it as a conversion electron; or used to create and emit a new particle (alpha particle or beta particle) from the nucleus.

The term “subject” or “patient” refers to a living organism, including both humans and animals, to which an agent is administered, the agent allowing visualisation of a specific molecular target or biomarker in the patient.

As used herein, the term “diagnosing” or grammatically equivalent wordings, means determining whether or not a subject suffers from a particular disease or disorder.

As used herein, “prognosing” or grammatically equivalent wordings, means determining whether or not a subject has a risk of developing a particular disease or disorder.

As used herein, “image-guided therapy” refers to the use of any form of medical imaging to plan, perform, and evaluate surgical procedures and therapeutic interventions.

“Bimodal label” as used herein refers to a detectable label comprising a fluorescent moiety and a radionuclide.

“Targeting moiety” or “targeting agent” as used herein refers to an agent possessing binding affinity for a specific molecular target or biomarker. In the current invention such “targeting moiety” or “targeting agent” is coupled to a detectable label to form a targeted tracer that can for instance be used for in vivo medical imaging.

### Description

Medical imaging is a technique and process that is used to visualize the inside of an organism's body (or parts and/or functions thereof), for clinical purposes (e.g. disease diagnosis, prognosis or therapy monitoring) or medical science (e.g. study of anatomy and physiology). Conjugates of targeting molecules and detectable labels can be used as tracers for such in vivo medical imaging. The specific accumulation of a detectable label in the tissue of interest, requires the stable conjugation of said detectable label to a targeting molecule such as an antibody, antibody-fragment, protein scaffold, peptide or small molecule, recognizing a specific biomarker.

Full-sized monoclonal antibodies (MAbs) have a number of disadvantages that have so far limited their effective use as targeting agents for tracers in the clinic. MAbs are macromolecules with a relatively poor penetration into solid and isolated tissues such as tumors. In addition, complete MAbs feature a long residence time in the body and a potential increase in background signals because of binding to Fc-receptors on non-target cells, making them less suitable for molecular imaging applications. Indeed, for imaging the most important properties of a tracer are: rapid interaction with the target, fast clearing of unbound molecules from the body and low non-specific accumulation, especially around the area of interest. These requirements have led to the development of a myriad of antibody derived probe formats, trying to combine specificity with a small size for favorable pharmacokinetics. Immunoglobulin single variable domains are such antibody derived molecules wherein the antigen binding site is present on, and formed by, a single immunoglobulin domain (which is different from conventional immunoglobulins or their fragments, wherein typically two immunoglobulin variable domains interact to form an antigen binding site).

For in vivo medical imaging research and practice, fluorophores operating in the NIR region are most often used, because of superior imaging characteristics compared to light in the visible spectrum. More specifically, the lower background and improved tissue penetration-a consequence of diminished scattering, endogenous fluorophore absorption and autofluorescence- makes NIR light more suited.

In the NIR region, a wide choice of fluorophores is available for conjugation. Appropriate fluorophores should be (photo) stable in vitro and in vivo, be soluble in aqueous environments, and their extinction coefficient and quantum yield should be as high as possible to maximize brightness. For the labeling of proteins and small molecule ligands for in vivo medical imaging, cyanine-based fluorescent dyes are by far the most widely investigated. Cyanine dyes typically consist of two heterocyclic nitrogen-containing rings connected by a polymethine bridge. Extension of the polymethine bridge to penta- and heptamethines, red-shifts the absorption and emission of the dyes. Dyes, e.g. IRDye800CW, have been rendered less hydrophobic by the inclusion of several charged groups such as sulfonic acids in their structure.

IRDye800CW-labeled immunoglobulin single variable domains have been investigated for use in the context of intraoperative cancer imaging in various mouse models. Although tumor lesions could be successfully visualized through NIR fluorescence imaging, it was also shown that IRDye800CW-labeled immunoglobulin single variable domains exhibit significant non-specific uptake in vivo, in particular in the liver.

Alternatively, zwitterionic dyes such as ZW800-1, FNIR-Tag or s775z with balanced surface charges in their structure have been shown to interact very little with serum proteins, resulting in less background signals as compared to IRDye800CW-labeled tracers. This has for instance been demonstrated for RGD peptides labeled with ZW800-1 or FNIR-Tag-labeled antibodies.

It is commonly accepted in the field that various fluorescent tracers have an unpredictable biodistribution profile, showing that coupling of various fluorophores to the same targeting molecule, results in different pharmacokinetic properties. This indicates that it is not merely the targeting molecule that contributes to the pharmacokinetic properties of the conjugate, but also the fluorophore (Baranski et al, Improving the Imaging Contrast of 68Ga-PSMA-11 by Targeted Linker Design: Charged Spacer Moieties Enhance the Pharmacokinetic Properties. Bioconjug Chem. 2017 Sep. 20;28(9): 2485-2492.; Buckle T et al, Tracers for Fluorescence-Guided Surgery: How Elongation of the Polymethine Chain in Cyanine Dyes Alters the Pharmacokinetics of a Dual-Modality c [RGDyK] Tracer. J Nucl Med. 2018June;59(6): 986-992.; Bunschoten et al, Tailoring Fluorescent Dyes To Optimize a Hybrid RGD-Tracer. Bioconjug Chem. 2016 May 18;27(5): 1253-8.; Bao et al, Charge and hydrophobicity effects of NIR fluorophores on bone-specific imaging. Theranostics. 2015 Mar. 1;5(6): 609-17.)

As such, unfortunately, so far it remains unpredictable what the impact of a chosen dye will be on the pharmacokinetic profile and targeting capabilities of a specific targeting moiety (or class of similar targeting agents) as the physicochemical characteristics of a dye will affect in an individual manner the overall molecular charge, charge distribution and hydrophobicity of a tracer, and thus its blood clearance, excretion pathway, specific and non-specific uptake, metabolization etc.

In a first aspect, the invention relates to a conjugate comprising an immunoglobulin single variable domain conjugated to one or more detectable labels, wherein at least one of said labels comprises a fluorescent moiety, said fluorescent moiety having a structure chosen from formula I or formula II:

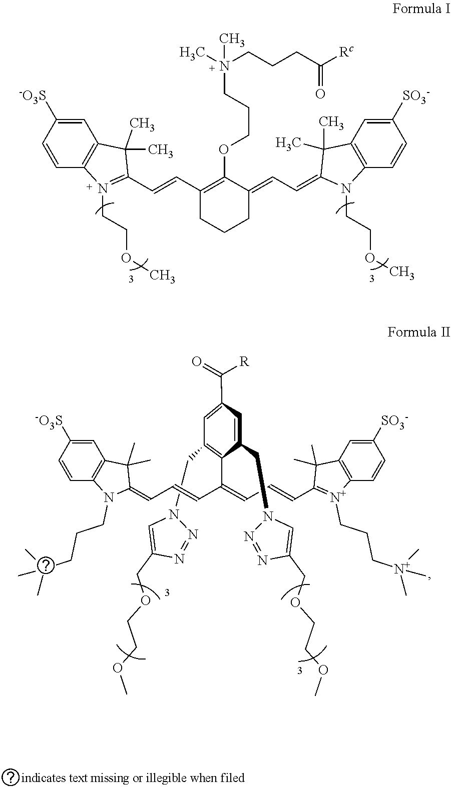

wherein R^{c} and R allow conjugation to the immunoglobulin single variable domain.

The inventors performed experiments using non-invasive 2D fluorescence imaging investigating whole body biodistribution and targeting abilities of the conjugates according to the current invention and of conjugates comprising the same immunoglobulin single variable domain coupled to either the previously discussed ZW800-1 or IRDye800CW fluorescent labels.

The inventors surprisingly found that conjugates according to the current invention comprising a fluorescent moiety having a structure chosen from formula I or formula II, show a clinically relevant biodistribution profile, with a fast biodistribution, clearance via the kidneys and very low background signals. Unexpectedly, conjugation of the fluorescent moieties having a structure according to Formula I or Formula II to the immunoglobin single variable domain does not negatively influence the in vivo biodistribution profile of the conjugates of the current invention. This is in contrast to IRDye800CW or ZW800-1-labeled immunoglobulin single variable domains. Immunoglobulin single variable domains labeled with IRDye800CW exhibit high-and long-lasting nonspecific signals in all tissues and organs following intravenous injection, and clinically relevant signal-to-background ratios are only reached after several hours. Furthermore, the liver is used as a major clearance route of the latter, giving high background signals and hindering the visualisation of conjugates targeted to the liver or surrounding cells. In addition, based on the acquired in vivo images, ZW800-1 labeled immunoglobulin single variable domains show low signal-to-background ratios, which remain low at all timepoints.

In contrast, conjugates according to the current invention comprising a fluorescent moiety having a structure chosen from formula I or formula II, show a clinically relevant biodistribution profile, with a fast biodistribution, clearance via the kidneys and very low background signals. In an embodiment, at least 50% of the in vivo administered dose of the conjugate according to the current invention is cleared via the kidneys. As a rule of thumb, a signal-to-background ratio of more than 1.5 is essential for efficient surgical guidance using tracers, and thus specificity of tracer accumulation and well-balanced accumulation and clearance kinetics are key.

In a preferred embodiment, the conjugate according to the current invention, when administered in vivo, enables, after irradiation with a quantity of light having a selected wavelength and selected intensity, a ratio of fluorescent target signal over fluorescent background signal higher than 1.5 when measured in organs other than the kidneys or when measured in organs and tissues which do not constitutively express the target of interest, measured within 6 hours after administration of between 0.1 and ×100 mg of said conjugate.

As clearance of the conjugate according to the current invention occurs via the kidneys, the fluorescent background signal is high there. The same holds true for healthy organs that constitutively express the target of interest.

Said one or more detectable labels are conjugated to said immunoglobulin single variable domain by means of a functional group. Such a functional group can be any reactive group known from the state of the art.

Functional groups, such as maleimide, NCS, NHS, TFP, PFP, alkyne, squaric acid or oligo-glycine nucleophiles, allow various conjugation methods. For instance, the isothiocyanate function (R-NCS) allows formation of stable thiourea bonds at alkaline pH with free amines. NHS, TFP, PFP and squaric acid are other examples of amine-reactive linkers. In another embodiment, site-specific conjugation methods are used, for instance using cysteine-maleimide chemistry, click chemistry or using enzyme-reactive chemistries (such as the use of sortase). Site-specific conjugation allows to further standardize the conjugate's composition if needed.

In an embodiment, R^{c} and R can be selected from —OH, an isothiocyanate group (-NCS), an NHS ester, an alkyne, a maleimide, a TFP ester, a PFP ester, an oligo-glycine nucleophile or a click chemistry reactive group.

In a preferred embodiment, said one or more detectable labels are conjugated to said immunoglobulin single variable domain by means of an amine-reactive crosslinker, for instance by activating the label with an NHS-ester. In the context of cGMP production for clinical translation, amine-reactive crosslinking chemistry is currently favorized because cysteine-tagged immunoglobulin single variable domains have a significantly reduced production yield, the risk exists that crucial intramolecular bridges are reduced, and cysteine/maleimide chemistry can be unstable over time in vivo.

The fluorescent moiety according to Formula I, also called “FNIR-Tag”, is net neutral. Because of this, binding to serum proteins such as HSA and AGP and membrane surfaces is minimized.

The fluorescent moiety according to Formula II, also called s775z, has a net charge of plus 1. However, said fluorescent moiety comprises two shielding arms that block undesired biological interactions and enhance photostability and can be classified as a sterically shielded heptamethine cyanine dye. The molecular design is based on the fact that a cyanine dye with a meso-Aryl substituent adopts a low-energy conformation with the plane of the aryl ring strongly rotated out the plane of the polyene. Adopting this molecular conformation alleviates steric crowding between the meso-Aryl ortho hydrogens and the proximal β hydrogens on the heptamethine chain. By simultaneously projecting two shielding arms directly over each face of the polyene, undesired biological interactions are blocked (such as binding to serum proteins) and photostability is enhanced.

As discussed above, the chosen dye has an important influence on the pharmacokinetic profile of a molecular tracer, and it is so far unpredictable what the impact will be for a specific class of molecular moiety.

Unexpectedly, conjugates according to the current invention, comprising an immunoglobulin single variable domain conjugated to such a FNIR-Tag or s775z label, show a clinically relevant biodistribution profile, with a fast biodistribution, clearance via the kidneys and very low background signals when administered in vivo.

As such, the conjugate according to the current invention having a fluorescent moiety with a structure chosen from formula I or formula II, displays a highly favourable pharmacokinetic profile.

Furthermore, immunoglobulin single variable domain-based tracers have attractive characteristics including rapid imaging (as soon as 1 h after in vivo injection), high specificity, deep tissue penetration, ease-of-generation and ease-of-use. One preferred class of immunoglobulin single variable domains corresponds to the VHH domains of naturally occurring heavy chain antibodies, also called “VHH domain sequences” or “single domain antibody fragment (sdAb)”.

Such VHH domain sequences can generally be generated or obtained by suitably immunizing a species of Camelid with a desired target, (i.e. so as to raise an immune response and/or heavy chain antibodies directed against a desired target), by obtaining a suitable biological sample from said Camelid (such as a blood sample, or any sample of B-cells), and by generating VHH sequences directed against the desired target, starting from said sample, using any suitable technique known per se. Such techniques will be clear to the skilled person. Alternatively, such naturally occurring VHH domains against the desired target can be obtained from naive libraries of Camelid VHH sequences, for example by screening such a library using the desired target or at least one part, fragment, antigenic determinant or epitope thereof using one or more screening techniques known per se. Such libraries and techniques are for example described in WO9937681, WO0190190, WO03025020 and WO03035694. Alternatively, improved synthetic or semi-synthetic libraries derived from naive VHH libraries may be used, such as VHH libraries obtained from naive VHH libraries by techniques such as random mutagenesis and/or CDR shuffling, as for example described in WO0043507. Yet another technique for obtaining VHH domain sequences directed against a desired target involves suitably immunizing a transgenic mammal that is capable of expressing heavy chain antibodies (i.e. so as to raise an immune response and/or heavy chain antibodies directed against a desired target), obtaining a suitable biological sample from said transgenic mammal (such as a blood sample, or any sample of B-cells), and then generating VHH domain sequences directed against the desired target starting from said sample, using any suitable technique known per se. For example, for this purpose, the heavy chain antibody-expressing mice and the further methods and techniques described in WO02085945 and in WO04049794 can be used.

A particularly preferred class of immunoglobulin single variable domains of the invention comprises VHH domain sequences with an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being. This can be performed in a manner known per se, which will be clear to the skilled person and on the basis of the prior art on humanization. Again, it should be noted that such humanized VHH domain sequences of the invention can be obtained in any suitable manner known per se and thus are not strictly limited to polypeptides that have been obtained using a polypeptide that comprises a naturally occurring VHH domain as a starting material. Humanized VHH domain sequences may have several advantages, such as a reduced immunogenicity, compared to the corresponding naturally occurring VHH domains. Such humanization generally involves replacing one or more amino acid residues in the sequence of a naturally occurring VHH with the amino acid residues that occur at the same position in a human VH domain, such as a human VH3 domain. The humanizing substitutions should be chosen such that the resulting humanized VHH domain sequences still retain the favourable properties of VHH domain sequences as defined herein. The skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand.

Also within the scope of the invention are natural or synthetic analogs, mutants, variants, alleles, homologs and orthologs of the immunoglobulin single variable domain of the invention as defined herein.

By means of non-limiting examples, a substitution may for example be a conservative substitution (as described herein) and/or an amino acid residue may be replaced by another amino acid residue. Thus, any one or more substitutions, deletions or insertions, or any combination thereof, that either improve the properties of the immunoglobulin single variable domain of the invention or that at least do not detract too much from the desired properties or from the balance or combination of desired properties of the immunoglobulin single variable domain of the invention (i.e. to the extent that the immunoglobulin single variable domain is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the immunoglobulin single variable domain thus obtained.

Further, depending on the host organism used to express the immunoglobulin single variable domain of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups.

Examples of modifications, as well as examples of amino acid residues within the immunoglobulin single variable domain sequence, that can be modified, methods and techniques that can be used to introduce such modifications and the potential uses and advantages of such modifications will be clear to the skilled person. For example, such a modification may involve the introduction (e.g. by covalent linking or in another suitable manner) of one or more functional groups, residues or moieties into or onto the immunoglobulin single variable domain, and in particular of one or more functional groups, residues or moieties that confer one or more desired properties or functionalities to the immunoglobulin single variable domain of the invention. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including single domain antibody fragments). Such functional groups may for example be linked directly (for example covalently) to an immunoglobulin single variable domain of the invention, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly (ethyleneglycol) (PEG) or derivatives thereof (such as methoxypoly (ethyleneglycol) or mPEG). Generally, any suitable form of pegylation can be used, such as the pegylation used in the art for antibodies and antibody fragments. In an embodiment, site-directed pegylation is used, in particular via a cysteine-residue. For example, for this purpose, PEG may be attached to a cysteine residue that naturally occurs in an immunoglobulin single variable domain of the invention, an immunoglobulin single variable domain of the invention may be modified so as to suitably introduce one or more cysteine residues for attachment of PEG, or an amino acid sequence comprising one or more cysteine residues for attachment of PEG may be fused to the N- and/or C-terminus of an immunoglobulin single variable domain of the invention, all using techniques of protein engineering known per se to the skilled person. Another, usually less preferred modification comprises N-linked or O-linked glycosylation, usually as part of co-translational and/or post-translational modification, depending on the host cell used for expressing immunoglobulin single variable domain of the invention.

The binding of conjugates or tracers to serum proteins is an important process that determines the activity and pharmacokinetics of the tracer in the body. After being distributed around the circulating system, tracers bind to serum proteins in varying degrees. In general, such binding is reversible, and an equilibrium exists between bound and free molecular forms. Unfortunately, the bound tracer can cause undesirable background signals by increased signal in blood, liver and other non-targeted tissues. Therefore tracer-serum protein binding is considered as an important property and needs to be optimized to allow efficient visualisation of target molecules.

In an aspect, the invention provides a conjugate comprising an immunoglobulin single variable domain conjugated to one or more detectable labels, wherein said conjugate exhibits less than 10 percent, more preferably less than 9 percent, more preferably less than 8 percent, more preferably less than 7 percent, more preferably less than 6 percent, more preferably less than 5 percent, more preferably less than 4 percent, more preferably less than 3 percent, more preferably less than 2 percent, more preferably less than 1 percent serum protein binding as measured by HPLC.

A person of ordinary skill will appreciate that elements of the aspects as described above are also applicable here. Consequently, all aspects of the present invention are related. All features and advantages as described in one of the aspects as described above, can relate to any of these aspects, even if they are described in conjunction with a specific aspect. As such, features and advantages of the immunoglobulin single variable domain as described above are also applicable here.

Said one or more detectable labels are conjugated to said immunoglobulin single variable domain by means of a functional group, such as an amine-reactive cross-linker or a sulfhydryl-reactive crosslinker.

In a more preferred embodiment, said one or more detectable labels are conjugated to said immunoglobulin single variable domain by means of an amine-reactive crosslinker, for instance by activating the label with an NHS-ester. In the context of cGMP production for clinical translation, amine-reactive crosslinking chemistry is currently favorized because cysteine-tagged immunoglobulin single variable domains have a significantly reduced production yield, the risk exists that crucial intramolecular bridges are reduced, and cysteine/maleimide chemistry can be unstable over time in vivo.

It is known that the properties of the detectable label can have an influence on the pharmacokinetic profile of a tracer conjugate when administered in vivo. For instance, the lipophilicity of the detectable label can influence nonspecific interactions with nontarget tissue and the amount of serum binding of the conjugate.

As discussed above, binding of the conjugate to serum proteins is unwanted as it influences the physical and functional properties of the conjugate.

In a preferred embodiment, at least one of said labels conjugated to the immunoglobulin single variable domain comprises a fluorescent moiety, said fluorescent moiety having a structure chosen from formula I or formula II:

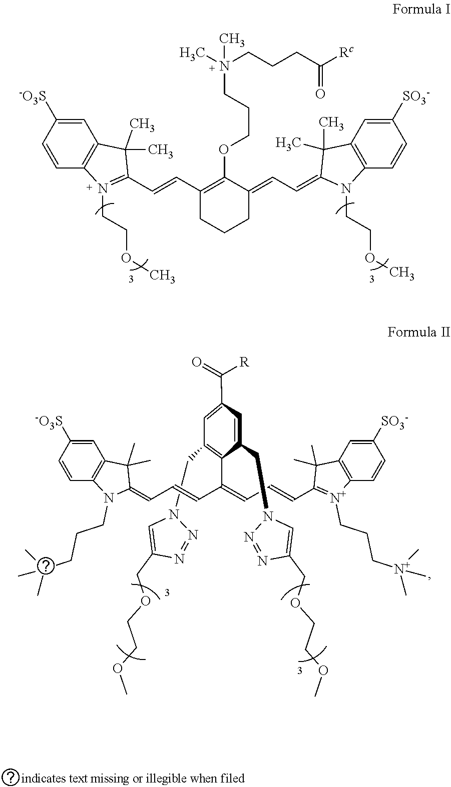

wherein R^{c} and R allow conjugation to the immunoglobulin single variable domain. As described above, said one or more detectable labels are conjugated to said immunoglobulin single variable domain by means of a functional group.

Such a functional group can be any reactive group known from the state of the art. In an embodiment, R^{c} and R can be selected from —OH, an isothiocyanate group (-NCS), an NHS ester, a squaric acid, an alkyne, a maleimide, a TFP ester, a PFP ester, an oligo-glycine nucleophile or a click chemistry reactive group.

Functional groups, such as maleimide, NCS, NHS, TFP, PFP, squaric acid, alkyne or oligo-glycine nucleophiles, allow various conjugation methods. For instance, the isothiocyanate function (R-NCS) allows formation of stable thiourea bonds at alkaline pH with free amines. NHS, TFP, squaric acid and PFP are other examples of amine-reactive linkers. In another embodiment, site-specific conjugation methods are used, for instance using cysteine-maleimide chemistry, click chemistry or using enzyme-reactive chemistries (such as the use of sortase). Site-specific conjugation allows to further standardize the conjugate's composition if needed.

In a preferred embodiment, the fluorescent moiety according to formula I exhibits an excitation maximum between 750 nm and 780 nm, more preferably between 760 and 770 nm, such as 765 nm. In a preferred embodiment, the fluorescent moiety according to formula I exhibits an emission maximum between 770 nm and 800 nm, more preferably between 785 and 795 nm, such as 788 nm.

In a preferred embodiment, the conjugate according to the current invention comprising a fluorescent moiety according to formula I exhibits an excitation maximum between 750 nm and 780 nm, more preferably between 760 and 770 nm. In a preferred embodiment, the conjugate according to the current invention comprising a fluorescent moiety according to formula I exhibits an emission maximum between 770 nm and 800 nm, more preferably between 785 and 795 nm.

In a preferred embodiment, the fluorescent moiety according to formula II exhibits an excitation maximum between 760 nm and 790 nm, more preferably between 770 and 780 nm, such as 775 nm. In a preferred embodiment, the fluorescent moiety according to formula II exhibits an emission maximum between 780 nm and 810nm, more preferably between 790 and 800 nm, such as 794 nm.

In a preferred embodiment, the conjugate according to the current invention comprising a fluorescent moiety according to formula II exhibits an excitation maximum between 760 nm and 790 nm, more preferably between 770 and 780 nm. In a preferred embodiment, the conjugate according to the current invention comprising a fluorescent moiety according to formula II exhibits an emission maximum between 790 nm and 810 nm, more preferably between 795 and 810 nm.

The inventors performed experiments using non-invasive 2D fluorescence imaging investigating whole body biodistribution and targeting abilities of the conjugates according to the current invention and of conjugates comprising the same immunoglobulin single variable domain coupled to either the previously discussed ZW800-1 or IRDye800CW fluorescent labels.

The inventors surprisingly found that conjugates according to the current invention comprising a fluorescent moiety having a structure chosen from formula I or formula II, show a clinically relevant biodistribution profile, with a fast biodistribution, clearance via the kidneys and very low background signals. Unexpectedly, conjugation of the fluorescent moieties having a structure according to Formula I or Formula II to the immunoglobin single variable domain does not negatively influence the in vivo biodistribution profile of the conjugates of the current invention. This is in contrast to IRDye800CW or ZW800-1-labeled immunoglobulin single variable domains. Immunoglobulin single variable domains labeled with IRDye800CW exhibit high-and long-lasting nonspecific signals in all tissues and organs following intravenous injection, and clinically relevant signal-to-background ratios are only reached after several hours. Furthermore, the liver is used as a major clearance route of the latter, giving high background signals and hindering the visualisation of conjugates targeted to the liver or surrounding cells. In addition, based on the acquired in vivo images, ZW800-1 labeled immunoglobulin single variable domains show low signal-to-background ratios, which remain low at all timepoints.

In contrast, conjugates according to the current invention comprising a fluorescent moiety having a structure chosen from formula I or formula II, show a clinically relevant biodistribution profile, with a fast biodistribution, clearance via the kidneys and very low background signals. In an embodiment, at least 50% of the in vivo administered dose of the conjugate according to the current invention is cleared via the kidneys. As a rule of thumb, a signal-to-background ratio of more than 1.5 is essential for efficient surgical guidance using tracers, and thus specificity of tracer accumulation and well-balanced accumulation and clearance kinetics are key.

In a preferred embodiment, the conjugate according to the current invention, when administered in vivo, enables, after irradiation with a quantity of light having a selected wavelength and selected intensity, a ratio of fluorescent target signal over fluorescent background signal higher than 1.5 when measured in organs other than the kidneys or when measured in organs and tissues which do not constitutively express the target of interest, measured within 6 hours after administration of between 0.1 and ×100 mg of said conjugate.

While the use of NIR fluorescent dyes has led to increased signal-to-background ratios and better overall depth penetration up to several millimeter in tissue, deeper lying, and hidden lesions could still be missed, for instance in the field of cancer imaging. To this end, the combination of NIR fluorescence and nuclear techniques in a single tracer is attractive. In the field of cancer surgery, the nuclear component can be used for preoperative imaging, as well as for intraoperative guidance using a gamma-detecting probe (coarse navigation), at which point, fluorescence would provide high resolution visual guidance for precise resection. In these types of surgeries, the primary goal is to attain complete removal of all cancerous tissue and obtain negative tumor resection margins. By minimizing the risk of leaving cancer cells behind, chances of recurrence are diminished and overall survival is improved.

In an embodiment, said one or more detectable labels comprises a bimodal label comprising a fluorescent moiety and a radionuclide.

In an embodiment, the radionuclide component of the conjugate is used for preoperative imaging. Such preoperative imaging allows to better plan the surgery as it provides useful information on the anatomical localization of the tumor lesions and presence of lymph node metastases and often involves SPECT or PET imaging. In another embodiment, the radionuclide component of the conjugate can provide additional guidance during the surgery itself or during ex vivo analysis of the resected tissues via gamma-ray, positron or beta-minus detection (probe or camera), or Cerenkov luminescence imaging.

In an embodiment, said conjugate comprising such a bimodal label is used for precise and sensitive intraoperative guidance during resection of tumors. SPECT/CT or PET/CT scans (for instance 1 h post-injection) could be used to localize the large tumor lesions. Subsequent opening of the abdominal cavity can allow in situ fluorescence-based visualization and fluorescence-guided removal of even submillimeter tumor lesions that were spread at the surface of the patient's peritoneum. Such a bimodal tracer allows detection of larger lesions via nuclear imaging, while fluorescence enables accurate removal of submillimeter lesions. In an embodiment, radioguided surgery solutions in the form of intraoperative gamma tracing can further help to further guide the surgeon towards cancerous lesions.

In an embodiment said radionuclide is chosen from the group of fluor 18 (¹⁸F), lutetium 177 (¹⁷⁷Lu), zirconium 89 (⁸⁹Zr), indium 111 (¹¹¹In), yttrium 90 (⁹⁰Y), copper 64 (⁶⁴Cu), gallium 67 (⁶⁷Ga), gallium 68 (⁶⁸Ga), technetium 99m (^{99m}Tc), iodium 123 (¹²³I), iodium 124 (¹²⁴I), iodium 125 (¹²⁵I), iodium 131 (¹³¹I).

To design a targeted tracer that can be detected with both modalities, i.e., a bimodal tracer, the most straightforward method would be by randomly conjugating a fluorophore and chelator/prosthetic group directly to the targeting moiety. This strategy has been used for antibodies or large antibody fragments. It does, however, lead to heterogeneous functionalization rates of the different labels, making it complex to standardize the tracer's composition. For targeting moieties such as small antibody fragments, peptides and small molecules that only have a limited number of conjugation sites, the conjugation of multiple labels can have a more pronounced detrimental effect on their functionality and biodistribution, leading to the introduction of single bimodal labels. The integration of both a fluorescent dye and a chelator for radioactive labeling into a single backbone structure has been pursued in a range of designs. One often bimodal label design is based on an amino acid backbone modified to bear a fluorophore, a chemical group for subsequent radiolabeling, and a reactive group for conjugation. A genetically encoded sequence at the terminus of the targeting moiety, onto which both labels can be site-specifically conjugated, forms an alternative.

In an embodiment, said bimodal label is a single bimodal label. In such a single bimodal label, the fluorophore and the radiolabel can be combined into a single structure. This is advantageous since a more homogenous tracer is obtained, whose biodistribution can be accurately determined via radioactivity. Furthermore, this is often the only possibility when working with smaller targeting moieties, such as immunoglobulin single variable domains, as multiple conjugation sites may not be available or are not desirable.

The immunoglobulin single variable domain can be for instance labeled using a trivalent amino-acid base backbone that integrates a fluorescent moiety and a group for subsequent radioactive labeling into a single structure, as well as a reactive group for conjugation to the immunoglobulin single variable domain. In a preferred embodiment, the average degree of labeling (DOL) is between 0.5 and 2 per immunoglobulin single variable domain.

Said group for radioactive labeling can be any reactive group known from the state of the art, such as a agent chelating (for instance DTPA (Diethylentriaminepentaacetic acid), DOTA (1,4,7,10-Tetraazacyclododecane-1,4,7,10-tetraacetic acid) or NOTA (1,4,7-Triazacyclononane-1,4,7-triacetic acid)) or a leaving group for fluorination or iodination (for instance quaternary ammonium, nitrogroups, triflate, tosylate, nosylate or mesylate).

In an embodiment, said immunoglobulin single variable domain is directed against and/or specifically binds to one or more clinically relevant targets in a body of a patient.

As used herein, the term “specifically binding to” refers to the ability of said immunoglobulin single variable domain to preferentially bind to a particular antigen that is present in a homogeneous mixture of different antigens and does not necessarily imply high affinity (as defined further herein). In certain embodiments, a specific binding interaction will discriminate between desirable and undesirable antigens in a sample, in some embodiments more than about 10 to 100-fold or more (e.g., more than about 1000- or 10,000-fold). The term “affinity”, as used herein, refers to the degree to which the immunoglobulin single variable domain binds to an antigen so as to shift the equilibrium of antigen and immunoglobulin single variable domain toward the presence of a complex formed by their binding. Thus, for example, where an antigen and immunoglobulin single variable domain are combined in relatively equal concentration, an immunoglobulin single variable domain of high affinity will bind to the available antigen so as to shift the equilibrium toward high concentration of the resulting complex. The dissociation constant is commonly used to describe the affinity between the immunoglobulin single variable domain and the antigenic target. Typically, the dissociation constant is lower than 10⁻⁵ M. Preferably, the dissociation constant is lower than 10⁻⁶ M, more preferably, lower than 10⁻⁷ M. Most preferably, the dissociation constant is lower than 10⁻⁸ M.

In an embodiment, said immunoglobulin single variable domain can be directed against and/or specifically bind to one or more targets or proteins specifically expressed by cell types belonging to a specific anatomical structure.

In another embodiment, said immunoglobulin single variable domain can be directed against and/or specifically bind to one or more targets being linked to a specific disease or pathology or to proteins specifically expressed by cell types involved in a specific disease or pathology. Activated inflammatory cells, such as macrophages, can contribute to the pathophysiology of disease in some instances.

In an embodiment, said immunoglobulin single variable domain can be directed against and/or specifically bind to one or more targets being linked to the development and progression of cancer (such as proliferation and survival of cancer cells and cancer metastasis), for instance human epidermal growth factor receptor type 2 (HER2), epidermal growth factor receptor (EGFR), Carcinoembryonic antigen (CEA), Prostate-Specific Membrane Antigen (PSMA), Folate Receptor a (FRa), Vascular Cell Adhesion Molecule-1 (VCAM-1), urokinase plasminogen activator receptor (uPAR), 5T2 multiple myeloma (MM) cell-produced M-protein or to proteins specifically expressed by cell types involved in one or more of the aforementioned processes, for instance the expression of macrophage mannose receptor (MMR) or Signal regulatory protein a (SIRPa) by tumor-associated macrophages in the tumor or the expression of fibroblast activation protein (FAP) by cancer-associated fibroblasts.

In an embodiment, said immunoglobulin single variable domain is directed against and/or specifically binds to EGFR. In an embodiment, said immunoglobulin single variable domain directed against and/or specifically binding to EGFR has following sequence: QVKLEESGGGSVQTGGSLRLTCAASGRTSRSYGMGWFRQAPGKEREFVSGISW RGDSTGYADSVKGRFTISRDNAKNTVDLQMNSLKPEDTAIYYCAAAAGSAWYGTLYEYDYW GQGTQVTVSS (SEQ ID NO: 1). Said immunoglobulin single variable domain having SEQ ID NO: 1 is also referred to as “7D12”. In an embodiment, said immunoglobulin single variable domain comprises an amino acid sequence consisting of polypeptides that have at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably 100% amino acid identity with SEQ ID NO: 1. In an embodiment, said immunoglobulin single variable domain comprises an amino acid sequence consisting of polypeptides that have 1, 2 or 3 amino acid difference with SEQ ID NO: 1.

In an embodiment, the immunoglobulin single variable domain directed against and/or specifically binding to EGFR (for instance having SEQ ID NO:1 or an immunoglobulin single variable domain comprising an amino acid sequence consisting of polypeptides that have at least 90% amino acid identity with SEQ ID NO: 1 or consisting of polypeptides that have 1, 2 or 3 amino acid difference with SEQ ID NO: 1) is linked with a tag, preferably a C-terminal tag, for instance to allow easy purification or site-specific labeling. Suitable tags are known from the art and include for instance affinity tags (such as Glutathione-S-transferase-tag, PolyHis tag, Biotin, Strep-tag, sortase-tag, glutamine-tag, unnatural amino acid tag, glycosylation-tag, etc.) or epitope tags (such as C-myc-tag, Human influenza hemagglutinin (HA)-tag, DDDK-tag or V-tag). In an embodiment, said immunoglobulin single variable domain directed against and/or specifically binding to EGFR is linked with a carboxy-terminal hexahistidine tag. In an embodiment, the immunoglobulin single variable domain having SEQ ID NO: 1 (“7D12”) is linked with a carboxy-terminal hexahistidine tag, said construct being referred to as “7D12.6His”. In another embodiment, the immunoglobulin single variable domain directed against and/or specifically binding to EGFR is not linked with a tag. In said instance, the immunoglobulin single variable domain having SEQ ID NO: 1 (“7D12”) is also referred to as “7D12NT” (“NT”=“No Tag”).

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Determining the percentage of sequence identity can be done manually, or by making use of computer programs that are available in the art. Examples of useful algorithms are PILEUP (Higgins & Sharp, CABIOS 5:151 (1989), BLAST and BLAST 2.0 (Altschul et al. J. Mol. Biol. 215:403 (1990). Software for performing BLAST analyses is publicly available through the National Center for Biotechnology Information (http://www.ncbi.nlm.nih.gov/).

In an embodiment, said immunoglobulin single variable domain is directed against and/or specifically binds to HER2. In an embodiment, said immunoglobulin single variable domain directed against and/or specifically binding to HER2 has following sequence: QVQLQESGGGSVQAGGSLKLTCAASGYIFNSCGMGWYRQSPGRERELVSRISG DGDTWHKESVKGRFTISQDNVKKTLYLQMNSLKPEDTAVYFCAVCYNLETYWGQGTQVTVS S (SEQ ID NO: 2). Said immunoglobulin single variable domain having SEQ.ID NO: 2 is also referred to as “2Rs15d”. In an embodiment, said immunoglobulin single variable domain comprises an amino acid sequence consisting of polypeptides that have at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably 100% amino acid identity with SEQ ID NO: 2. In an embodiment, said immunoglobulin single variable domain comprises an amino acid sequence consisting of polypeptides that have 1, 2 or 3 amino acid difference with SEQ ID NO: 2.

In an embodiment, the immunoglobulin single variable domain directed against and/or specifically binding to HER2 (for instance having SEQ ID NO:2 or an immunoglobulin single variable domain comprising an amino acid sequence consisting of polypeptides that have at least 90% amino acid identity with SEQ ID NO: 2 or consisting of polypeptides that have 1, 2 or 3 amino acid difference with SEQ ID NO: 2) is linked with a tag, preferably a C-terminal tag, for instance to allow easy purification or site-specific labeling. Suitable tags are known from the art and are discussed above. In an embodiment, said immunoglobulin single variable domain directed against and/or specifically binding to HER2 is linked with a carboxy-terminal hexahistidine tag. In an embodiment, the immunoglobulin single variable domain having SEQ ID NO: 2 (“2Rs15d”) is linked with a carboxy-terminal hexahistidine tag, said construct being referred to as “2Rs15d.6His”. In another embodiment, the immunoglobulin single variable domain directed against and/or specifically binding to HER2 is not linked with a tag. In said instance, the immunoglobulin single variable domain having SEQ ID NO: 2 (“2Rs15d”) is also referred to as “2Rs15dNT” (“NT”=No Tag).

In an embodiment, said immunoglobulin single variable domain is directed against and/or specifically binds to CEA. In an embodiment, said immunoglobulin single variable domain directed against and/or specifically binding to CEA has following sequence: QVQLVESGGGSVQAGGSLRLSCAASGDTYGSYWMGWFRQAPGKEREGVAAIN RGGGYTVYADSVKGRFTISRDTAKNTVYLQMNSLRPDDTADYYCAASGVLGGLHEDWFNYW GQGTQVTVSS (SEQ ID NO: 3). Said immunoglobulin single variable domain having SEQ ID NO: 3 is also referred to as “CEA5”. In an embodiment, said immunoglobulin single variable domain comprises an amino acid sequence consisting of polypeptides that have at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably 100% amino acid identity with SEQ ID NO: 3. In an embodiment, said immunoglobulin single variable domain comprises an amino acid sequence consisting of polypeptides that have 1, 2 or 3 amino acid difference with SEQ ID NO: 3.

In an embodiment, the immunoglobulin single variable domain directed against and/or specifically binding to CEA (for instance having SEQ ID NO: 3 or an immunoglobulin single variable domain comprising an amino acid sequence consisting of polypeptides that have at least 90% amino acid identity with SEQ ID NO: 3 or consisting of polypeptides that have 1, 2 or 3 amino acid difference with SEQ ID NO: 3) is linked with a tag, preferably a C-terminal tag, for instance to allow easy purification or site-specific labeling. Suitable tags are known from the art and are discussed above. In an embodiment, said immunoglobulin single variable domain directed against and/or specifically binding to CEA is linked with a carboxy-terminal hexahistidine tag. In an embodiment, the immunoglobulin single variable domain having SEQ ID NO: 3 (“CEA5”) is linked with a carboxy-terminal hexahistidine tag, said construct being referred to as “CEA5.6His”. In another embodiment, the immunoglobulin single variable domain directed against and/or specifically binding to CEA is not linked with a tag. In said instance, the immunoglobulin single variable domain having SEQ ID NO:3 (“CEA5”) is also referred to as “CEA5NT” (“NT”=No Tag).

In an embodiment, said immunoglobulin single variable domain is directed against and/or specifically binds to 5T2 multiple myeloma (MM) cell-produced M-protein. In an embodiment, said immunoglobulin single variable domain directed against and/or specifically binding to 5T2 multiple myeloma (MM) cell-produced M-protein has following sequence: QVQLQESGGGSVQAGGSLRLSCAASGDTGYMGWFRQAPGKEREGVAVINSDSGVGSTYYA DSVKGRFTISRDNAKNTVYLQMNSLKPEDTAIYYCAAGHFSDYVSPWTWREIYRYNVWGQG TQVTVSS (SEQ ID NO: 4). Said immunoglobulin single variable domain having SEQ ID NO: 4 is also referred to as “R3B23”. In an embodiment, said immunoglobulin single variable domain comprises an amino acid sequence consisting of polypeptides that have at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably 100% amino acid identity with SEQ ID NO: 4. In an embodiment, said immunoglobulin single variable domain comprises an amino acid sequence consisting of polypeptides that have 1, 2 or 3 amino acid difference with SEQ ID NO: 4.

In an embodiment, the immunoglobulin single variable domain directed against and/or specifically binding to 5T2 multiple myeloma (MM) cell-produced M-protein (for instance having SEQ ID NO:4 or an immunoglobulin single variable domain comprising an amino acid sequence consisting of polypeptides that have at least 90% amino acid identity with SEQ ID NO: 4 or consisting of polypeptides that have 1, 2 or 3 amino acid difference with SEQ ID NO: 4) is linked with a tag, preferably a C-terminal tag, for instance to allow easy purification or site-specific labeling. Suitable tags are known from the art and are discussed above. In an embodiment, said immunoglobulin single variable domain directed against and/or specifically binding to 5T2 multiple myeloma (MM) cell-produced M-protein is linked with a carboxy-terminal hexahistidine tag. In an embodiment, the immunoglobulin single variable domain having SEQ ID NO: 4 (“R3B23”) is linked with a carboxy-terminal hexahistidine tag, said construct being referred to as “R3B23.6His”. In another embodiment, the immunoglobulin single variable domain directed against and/or specifically binding to 5T2 multiple myeloma (MM) cell-produced M-protein is not linked with a tag. In said instance, the immunoglobulin single variable domain having SEQ ID NO: 4 (“R3B23”) is also referred to as “R3B23NT” (“NT”=No Tag).

In an embodiment, said immunoglobulin single variable domain is directed against and/or specifically binds to uPAR. In an embodiment, said immunoglobulin single variable domain directed against and/or specifically binding to uPAR has following sequence: QVQLQESGGGLVQSGGSLRLSCAVSGIGFVYYAMRWWRQAPGKERELVAGITG ASGNTNYAESVKDRFTISRDNASNTVYLQMNSLKPEDTAVYYCNAAPLIRFGPPKDYWGQGT QVTVSS (SEQ ID NO: 5). Said immunoglobulin single variable domain having SEQ

ID NO: 5 is also referred to as “uPAR13”. In an embodiment, said immunoglobulin single variable domain comprises an amino acid sequence consisting of polypeptides that have at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably 100% amino acid identity with SEQ ID NO: 5. In an embodiment, said immunoglobulin single variable domain comprises an amino acid sequence consisting of polypeptides that have 1, 2 or 3 amino acid difference with SEQ ID NO: 5.

In an embodiment, the immunoglobulin single variable domain directed against and/or specifically binding to uPAR (for instance having SEQ ID NO: 5 or an immunoglobulin single variable domain comprising an amino acid sequence consisting of polypeptides that have at least 90% amino acid identity with SEQ ID NO: 5 or consisting of polypeptides that have 1, 2 or 3 amino acid difference with SEQ ID NO: 5) is linked with a tag, preferably a C-terminal tag, for instance to allow easy purification or site-specific labeling. Suitable tags are known from the art and are discussed above. In an embodiment, said immunoglobulin single variable domain directed against and/or specifically binding to uPAR is linked with a carboxy-terminal hexahistidine tag. In an embodiment, the immunoglobulin single variable domain having SEQ ID NO: 5 (“uPAR13”) is linked with a carboxy-terminal hexahistidine tag, said construct being referred to as “uPAR13.6His”. In another embodiment, the immunoglobulin single variable domain directed against and/or specifically binding to uPAR is not linked with a tag. In said instance, the immunoglobulin single variable domain having SEQ ID NO: 5 (“uPAR13”) is also referred to as “uPAR13NT” (“NT”=“No Tag”).

In an embodiment, said immunoglobulin single variable domain can be directed against and/or specifically bind to one or more targets being linked to the development and progression of cardiovascular diseases or to proteins specifically expressed by cell types involved in one or more of the aforementioned processes.

In an embodiment, said immunoglobulin single variable domain can be directed against and/or specifically bind to one or more targets being linked to the development and progression of inflammatory disorders or diseases or to proteins specifically expressed by cell types involved in one or more of the aforementioned processes.

In an embodiment, said inflammatory disorder or disease is selected from the group consisting of: ulcerative colitis, rheumatoid arthritis, pulmonary fibrosis, atherosclerosis, multiple sclerosis, lupus erythematosus, psoriasis, osteomyelitis, Crohn's disease, graft versus host disease (GVHD), fibromyalgia, osteoarthritis, sarcoidosis, systemic sclerosis, Sjogren's syndrome, inflammations of the skin (e.g., psoriasis), glomerulonephritis, proliferative retinopathy, restenosis, and chronic inflammations.

The present invention also provides pharmaceutical formulations or compositions, both for veterinary and for human medical use, which comprise the conjugate of the invention with one or more pharmaceutically acceptable carriers.

The carrier(s) must be pharmaceutically acceptable in the sense of being compatible with the other ingredients of the formulation and not unduly deleterious to the recipient thereof. Such carriers are known in the art. In a preferred embodiment, said pharmaceutical composition is a sterile, non-pyrogenic formulation.

The conjugate and pharmaceutical compositions comprising said conjugate of the present invention can be administered by an appropriate route. Suitable routes of administration include, but are not limited to, orally, intraperitoneally, subcutaneously, intramuscularly, topically e.g. transdermally, rectally, sublingualis, intravenously, buccally, or inhalationally.

In some embodiments, the pharmaceutical compositions of the invention contain a pharmaceutically acceptable excipient suitable for rendering the compound or mixture administrable orally, parenterally, intravenously, intradermally, intramuscularly or subcutaneously, rectally, via inhalation or via buccal administration, or transdermally.

In some embodiments, the pharmaceutical compositions can be administered through urogenital routes to reach to targets, such as internal organs, topical lesions, or organs can be accessed by instillation (such as urinary bladder, vaginal cannel) more effectively and efficiently.

The conjugate can be admixed or compounded with a conventional, pharmaceutically acceptable excipient. It will be understood by those skilled in the art that a mode of administration, vehicle, excipient or carrier conventionally employed and which is inert with respect to the conjugate can be utilized for preparing and administering the pharmaceutical compositions of the present invention. The formulations of the present invention for use in a subject comprise the conjugate, together with one or more acceptable excipients, and optionally therapeutic agents. The excipient must be “acceptable” in the sense of being compatible with the other ingredients of the formulation and not deleterious to the recipient thereof.

The pharmaceutical formulations may be conveniently made available in a unit dosage form, whereby such formulations may be prepared by any of the methods generally known in the pharmaceutical arts. The combination of the conjugate with the one or more adjuvants is then physically treated to present the formulation in a suitable form for delivery (e.g., forming an aqueous suspension, by entrapping the drug in liposomes or microemulsions which are compatible with body tissues).

Since administration of parenteral dosage forms typically bypasses the patient's natural defenses against contaminants, parenteral dosage forms can be sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Adjuvants or accessory ingredients for use in the formulations of the present invention can include any pharmaceutical ingredient commonly deemed acceptable in the art, such as mixtures, buffers, solubility enhancers, and the like. Suitable vehicles that can be used to provide parenteral dosage forms of the compounds of the invention include, without limitation: sterile water; water for injection USP; saline solution; phosphate buffered saline; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a compound of the invention as disclosed herein can also be incorporated into the parenteral dosage forms of the disclosure, including conventional and controlled-release parenteral dosage forms.

Formulations for parenteral administration include aqueous and non-aqueous sterile injection solutions, which may further contain additional agents, such as anti-oxidants, buffers, bacteriostats, and solutes, which render the formulations isotonic with the blood of the intended recipient. In an embodiment, the composition comprises additives that reduce unwanted retention of the conjugate in the body, such as positively charged amino acids that have been shown to reduce kidney retention. The formulations may include aqueous and non-aqueous sterile suspensions, which contain suspending agents and thickening agents.

Injectable preparations, for example, sterile injectable aqueous or oleaginous suspensions can be formulated according to the known art using suitable dispersing or wetting agents and suspending agents. The sterile injectable preparation can also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

The present invention also provides convenient pharmaceutical kits. Such kits can comprise a conjugate of the invention and, typically, a pharmaceutically acceptable carrier. The kit can also further comprise conventional kit components, such as needles for use in injecting the compositions, one or more vials for mixing the composition components, and the like, as are apparent to those skilled in the art. In addition, instructions, either as inserts or as labels, indicating quantities of the components, guidelines for mixing the components, and protocols for administration, can be included in the kits.

In a further aspect, the invention relates to aforementioned conjugate or composition for use in in vivo medical imaging, preferably for prognostic, diagnostic and/or interventional purposes.

As used herein, the term “medical imaging” refers to the technique and process that is used to visualize the inside of an organism's body (or parts and/or functions thereof), for clinical purposes (e.g. disease diagnosis, prognosis or therapy monitoring) or medical science (e.g. study of anatomy and physiology). Examples of medical imaging methods include invasive techniques, such as intravascular ultrasound (IVUS), as well as non-invasive techniques, such as optical imaging, magnetic resonance imaging (MRI), ultrasound (US) and nuclear imaging. Examples of nuclear imaging include positron emission tomography (PET) and single photon emission computed tomography (SPECT). Medical optical imaging is the use of light as an investigational imaging technique for medical applications and includes for instance fluorescence-based techniques.

In a preferred embodiment, aforementioned conjugate or composition is used in the diagnosis, prognosis and/or image-guided therapy of a disease or pathology.

The conjugate or composition of the current invention allows for an improved pharmacokinetic profile compared to other tracers currently known in the field of medical imaging. As such, a more accurate diagnosis, prognosis and therapy can be obtained by the use of the conjugate of the current invention.

In a further preferred embodiment, aforementioned conjugate or composition is used in the diagnosis, prognosis or image-guided therapy of cancer. Said cancer is preferably selected from prostate cancer, liver cancer, head and/or neck cancer, brain cancer, skin cancer, bladder cancer, ovarian cancer, uterus cancer, lung cancer, breast cancer, sarcoma, cervix cancer, vulva cancer, hematologic cancers, renal cancer, bone cancer and/or gastrointestinal cancers.

In an embodiment, said immunoglobulin single variable domain is directed against and/or specifically binds to a tumor-associated antigen (also called a “solid tumor-specific antigen”, a “tumor-specific antigen”, “tumor antigen”, “target protein present on and/or specific for a (solid) tumor”, “tumor-specific target (protein)”. Such a tumor-associated antigen includes any protein which is present only on tumor cells and not on any other cell, or any protein, which is present on some tumor cells and also on some normal, healthy cells. Non-limiting examples of tumor antigens include tissue differentiation antigens, mutant protein antigens, oncogenic viral antigens, cancer-testis antigens and vascular or stromal specific antigens. It is expected that the immunoglobulin single variable domain as disclosed herein will bind to at least to those analogs, variants, mutants, alleles, parts and fragments of the tumor-associated antigen that (still) contain the binding site, part or domain of the natural tumor antigen to which those immunoglobulin single variable domains bind.

In an embodiment, said immunoglobulin single variable domain is directed against and/or specifically binds to proteins specifically expressed by tumor-associated cell types, such as macrophage mannose receptor (MMR) or Signal regulatory protein a (SIRPa) expressed by tumor-associated macrophages or fibroblast activation protein (FAP) expressed by tumor-associated fibroblasts.

In the context of the present invention, prognosing an individual suffering from or suspected to suffer from cancer refers to a prediction of the survival probability of an individual having cancer or the relapse risk which is related to the invasive or metastatic behavior (i.e. malignant progression) of tumor tissue or cells.

In an embodiment, said prognosis comprises cancer staging. Cancer staging is determined based on the size of the cancer and if or to what extent the cancer has spread to other parts of the patient's body. The conjugate of the current invention allows for an improved pharmacokinetic profile compared to other tracers currently known in the field of medical imaging. As such, a more accurate prognosis (including a more accurate cancer stage) can be determined. This allows to identify the most optimal treatment (for instance certain ongoing clinical trials) and improves the disease outcome (e.g. death, chance of recovery, recurrence).

In an embodiment, the use of the conjugate or composition of the current invention is used in preoperative imaging. Preoperative imaging findings support lesion identification during the intervention. The value of knowing where lesions are located during surgical procedures in relation to the patient's anatomic context is especially eminent when resections are performed in complex anatomies (e.g., the pelvic area).

In an embodiment of the invention, aforementioned conjugate or composition is used in endoscopic or intravascular imaging. Such imaging techniques can for instance be used for an improved prognosis and diagnosis of a disease or pathology.

In an embodiment, aforementioned conjugate or composition is used in endoscopic imaging for the detection of cancerous or precancerous lesions. White light endoscopy (WLE) has been the gold standard for the detection of lesions in the gastrointestinal tract. Despite the efficacy of current endoscopy, small and inconspicuous lesions or serrated adenomas tend to be flat and do not exhibit detectable discoloration relative to normal tissue in the three reflectance color channels (red/green/blue, RGB) used in WLE. This makes the detection of disease and the acquisition of targeted biopsies with conventional WLE a challenging issue. The use of the conjugate or composition of the current invention allows to detect inflammatory or (pre) cancerous lesions that might not have been detected using WLE, permitting more targeted biopsies and improving diagnostic performance.

In an embodiment, aforementioned conjugate or composition is used in intravascular imaging. In an embodiment said conjugate or composition is used in intraoperative intravascular imaging allowing localization and characterization of atherosclerotic lesions.

In an embodiment, aforementioned conjugate or composition is used in image-guided therapy of a disease or pathology.

Image-guided therapy refers to the use of any form of medical imaging to plan, perform, and evaluate surgical procedures and therapeutic interventions.

While the number of specific procedures that use image-guidance is growing, these procedures comprise two general categories: traditional surgeries that become more precise through the use of imaging and newer procedures that use imaging and special instruments to treat conditions of internal organs and tissues via minimally invasive surgery.

The most commonly used modalities of image-guided therapy include Magnetic Resonance Imaging (MRI) and Computed Tomography (CT). Image-guided therapy can also be supported by ultrasound, angiography, surgical navigation equipment, tracking tools, and integration software.

In an embodiment, aforementioned conjugate or composition is used in intraoperative image-guided surgery. A recent trend is the usage of fluorescence imaging during surgery, which is a real-time, sensitive, contact-free, relatively cheap, and non-ionizing technique that can easily be implemented within the surgical routine. Fluorescence imaging can provide anatomical, functional, or molecular information after administration of a fluorescent contrast agent, either through direct real-time imaging of the surgical field, or by intraoperative optical specimen mapping. Image-guidance help to make surgeries less invasive and more precise, which can lead to better survival, less complications, less morbidity, better quality of life, shorter hospital stays and fewer repeated procedures.

In an embodiment, aforementioned conjugate or composition is used in intraoperative image-guided surgery in cancer therapy. Surgery, in combination with or without chemo and/or radiotherapy, remains the most recommended treatment with curative intent for many localized tumors. In these types of surgeries, the primary goal is to attain complete removal of all cancerous tissue and obtain negative tumor resection margins. By minimizing the risk of leaving cancer cells behind, chances of recurrence are diminished and overall survival is improved. As visual inspection through the surgeon's eyes (open surgeries) or through color video (laparoscopic interventions) is often insufficient.

The conjugate or composition according to the current invention allows for real-time and sensitive information regarding the location of tumor lesions, allowing the detection of occult tumor lesions, accurate and real-time definition of tumor margins, and assessment of the presence of locoregional LN metastases. As such, the conjugate or composition according to the current invention allows for complete removal of all cancerous tissue and obtaining negative tumor resection margins, a major step forward in preventing over-and under-treatment, and personalizing the surgical treatment of many cancer patients.

In an embodiment, aforementioned conjugate or composition is used in pre-and posttherapy assessment.

In another embodiment, aforementioned conjugate or composition is used in non-surgical image-guided therapy, such as thermal ablation therapy. Ablation therapy relies on the principle that the tumor is not removed (like in a surgical resection) but rather destroyed in place by elevating the temperature within the tumorous tissues above a lethal threshold. The conjugate or composition of the current invention allows to accurately localize the tumor tissues that need to be ablated. Minimally invasive thermal ablation procedures are particularly well tolerated by the patients and require reduced hospital stays. In an embodiment, these image-guided ablation devices can be based on focused ultrasound (also called FUS or HIFU).

In a preferred embodiment, a dose of aforementioned conjugate or composition is administered to a patient, preferably a human patient, said dose ranging from 0.1 mg to 100 mg.

The useful dosage to be administered and the particular mode of administration will vary depending upon such factors as age, weight, and particular region to be visualised, the diagnostic use contemplated, and the form of the formulation, for example, suspension, emulsion, microsphere, liposome, or the like, as will be readily apparent to those skilled in the art.

In an embodiment, the conjugate or composition according to the current invention, when administered in vivo, or aforementioned use of said conjugate of composition, enables, after irradiation with a quantity of light having a selected wavelength and selected intensity, a ratio of fluorescent target signal over fluorescent background signal higher than 1.5 when measured in organs other than the kidneys or when measured in organs and tissues which do not constitutively express the target of interest, measured within 6 hours after administration of between 0.1 and ×100 mg of said conjugate.

The invention is further described by the following non-limiting examples which further illustrate the invention, and are not intended to, nor should they be interpreted to, limit the scope of the invention.

### EXAMPLES AND/OR DESCRIPTION OF FIGURES

The amino acid sequences of the sdAbs (7D12, 2Rs15d, CEA5, R3B23 and uPAR13) used during the following examples are listed above, having SEQ ID NOS: 1-5,respectively), the sdAbs are either linked to a carboxy-terminal hexahistidine tag (7D12.6His, CEA5.6His, R3B23.6His and uPAR13.6His) or not (2Rs15dNT).

### Example 1: Comparative Study Showing Improved Pharmacokinetics of the Conjugate According to the Current Invention

In this study, the aim is to investigate whether NIR dyes, such as IRDye800CW, ZW800-1, FNIR-Tag and s775z, can be used for the design of fluorescent single domain antibody (sdAb) tracers, including conjugates according to the current invention, with relevant in vivo biodistribution profiles.

### Material and Methods

Fluorescent Labeling of sdAbs

IRDye800CW and ZW800-1 were bought from LiCOR or Curadel, respectively. The FNIR-Tag (Luciano, M. P., et al., ACS Chem Biol, 2019. 14(5): p. 934-940) and s775z (Li, D. H., C. L. Schreiber, and B. D. Smith, Angew Chem Int Ed Engl, 2020. 59(29): p. 12154-12161) were gifted by respectively Prof. Martin J. Schnermann (Chemical Biology Laboratory, Center for Cancer Research, National Cancer Institute, United States) and Prof. Bradley Smith (Department of Chemistry and Biochemistry, University of Notre Dame, Notre Dame, IN, USA). The previously developed anti-HER2 sdAb 2Rs15dNT (Vaneycken, I., et al., FASEB J, 2011. 25(7): p. 2433-46 and Xavier, C., et al., J Nucl Med, 2013. 54(5): p. 776-84) and anti-EGFR sdAb 7D12.6His (Gainkam, L. O., et al., J Nucl Med, 2008. 49(5): p. 788-95) were labeled with the four different dyes. The anti-CEA sdAb CEA5.6His (Vaneycken, I., et al., J Nucl Med, 2010. 51(7): p. 1099-106) was labeled with FNIR-Tag and s775z according to an analogous protocol. Briefly, a 3-5× molar excess of fluorophore was added to the sdAbs in 0.1M K₂HPO₄ at pH 8.3-8.5. After 2 h incubation at room temperature, the conjugates were purified using size exclusion chromatography (SEC) on a Superdex75 Increase 10/300 GL column in phosphate buffered saline (PBS) (0.8 mL/min), and relevant fractions were collected to attain an average degree-of-labeling (DOL) of 1 fluorophore per sdAb. Analytical SEC for quality control was performed on a Superdex75 5/150 GL colum in with PBS as running buffer at 0.3 mL/min). The excitation and emission profiles of the resulting tracers were analysed with a fluorometer at a concentration of 1 μM (Shimadzu RF-6000).

In Vitro Serum Protein Binding 50 μg of fluorescent-labeled sdAb was added to 200 μL of human serum (Merck) at 37° C. The extent to which the sdAb binds to serum proteins was subsequently assessed via SEC on a Superdex75 5/150 GL column using PBS as running buffer at 0.3 mL/min. Serum proteins and the fluorescently-labeled sdAb were detected by measuring absorbance at respectively 280 nm and 775 nm.

### In Vivo Biodistribution and Tumor Targeting

For the in vivo imaging experiments Crl: NUFoxn1NU (Ncr) mice from Charles River were used (n=4 per group). They were kept in IVC cages and received water and food (Teklad 2016 low fluorescent diet) ad libitum. The mice were inoculated subcutaneously with 2×10⁶ FaDu (EGFR⁺ squamous cell carcinoma), SKOV3 (HER2⁺ ovarian cancer cell line) or BxPC3 (CEA⁺ pancreatic cancer cell line) cells on the right flank. After 10 days, they developed a tumor with a size of approximately 400 mm³.

2 nmol of fluorescent-labeled sdAb dissolved in saline was injected intravenously via the tail vein under isoflurane anaesthesia (2-3%, 0.5-1 mL/L flow rate). At various time points after injection (1 h, 3 h, 6 h, 12 h and 24 h), fluorescent images were acquired from the dorsal and ventral side of anesthetized mice using the FluoBeam800 (Fluoptics, Grenoble, France) (excitation at 780 nm, and detection of fluorescent light emitted with wavelengths over 820 nm). Images were taken with different exposure times in a dark room. After the last timepoint, mice were killed by cervical dislocation and organs of interest (tumour, liver, muscle, spleen, intestine, pancreas, stomach, heart, lungs and kidneys) were collected and imaged again ex vivo. An additional group of mice was included for dissection at 1 h post-injection.

### Image Analysis and Statistical Analysis

The fluorescent signal in different organs and tissues was quantified based on the images acquired in vivo and ex vivo using the software ImageJ. Region of interests (ROIs) were drawn on the organs/tissues and the average fluorescent signal was measured. In addition, tumor-to-organ ratios and liver-to-muscle ratios were calculated and put into a graphical representation using Prism 8 (GraphPad Software, San Diego, CA). Statistical comparisons for tumor-to-background organ between groups were performed using ANOVA tests with correction for multiple comparisons (Prism 8, GraphPad Software, San Diego, CA). Significance level was set at 0.05 (*p<0.05, ** p<0.01, *** p<0.001, **** p<0.0001).

### Results

### Fluorescent Labeling and Serum Protein Binding

The anti-EGFR and anti-HER2 sdAbs were successfully labeled with IRDye800CW, ZW800-1, FNIR-Tag or s775z (FIGS. 1A, A). The anti-CEA sdAbs were successfully labeled with FNIR-Tag or s775z. The resulting solutions had a DOL ranging between 1.0 and 1.3 on average and all tracers retained a similar excitation and emission profile as the unconjugated fluorescent dyes (FIGS. 1B, 2B, Table 1). IRDye800CW-labeled sdAbs exhibited more than 30% of binding to serum proteins (FIGS. 1C, 2C), while no serum protein binding was seen for the other fluorescently-labeled sdAbs.

TABLE 1
Dye per protein ratio and excitation/emission maxima
for the different fluorescently-labeled sdAbs

| | IRDye800CW | | ZW800-1 | | FNIR-Tag | | s775z | |
|---|---|---|---|---|---|---|---|---|
| | | Ex/Em | | Ex/Em | | Ex/Em | | Ex/Em |
| | DOL | (nm) | DOL | (nm) | DOL | (nm) | DOL | (nm) |
| Anti-HER2 2Rs15dNT | 1.0 | 781/798 | 1.0 | 774/793 | 1.0 | 765/792 | 1.0 | 782/803 |
| Anti-EGFR 7D12.6His | 1.0 | 777/797 | 1.0 | 768/793 | 1.0 | 765/790 | 1.0 | 779/802 |
| Anti-CEA CEA5.6His | n.a. | n.a. | n.a. | n.a. | 1.3 | 766/793 | 1.0 | 776/806 |

### In Vivo Biodistribution and Tumor Targeting

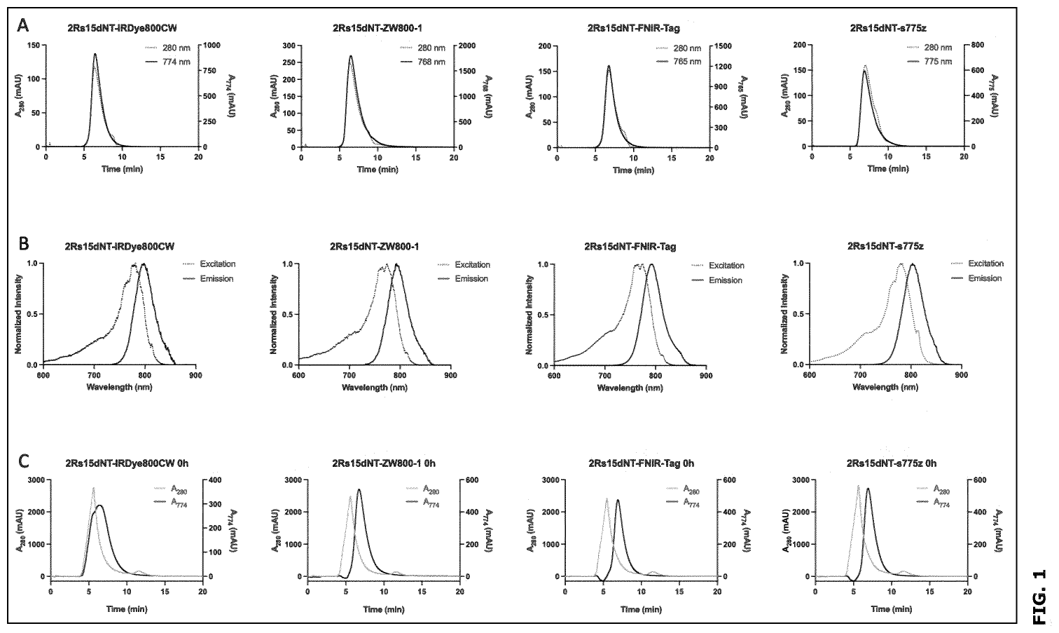

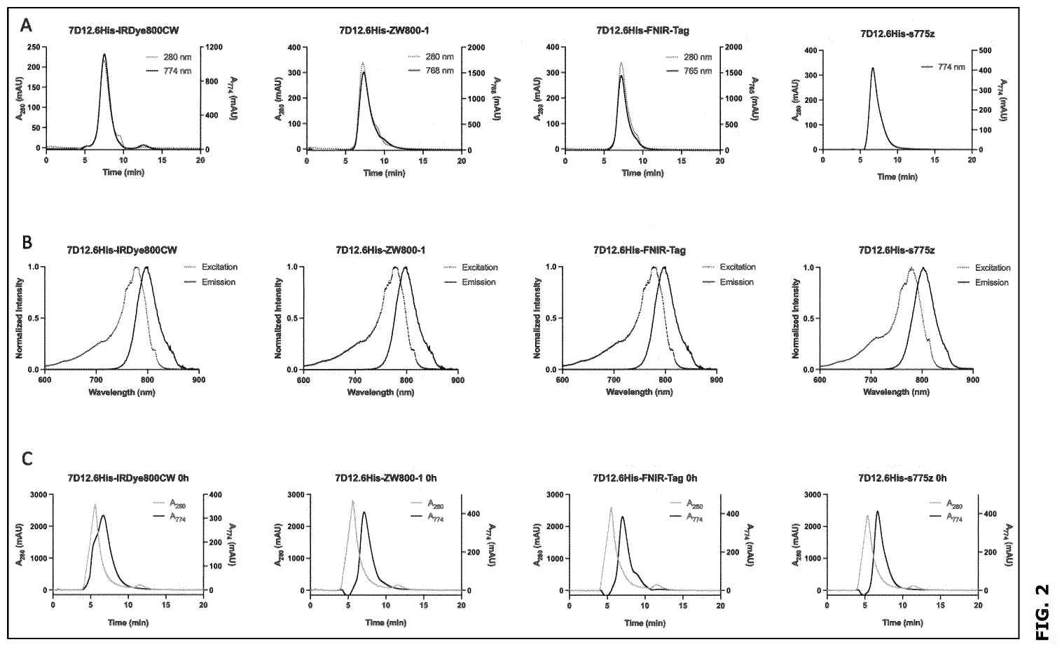

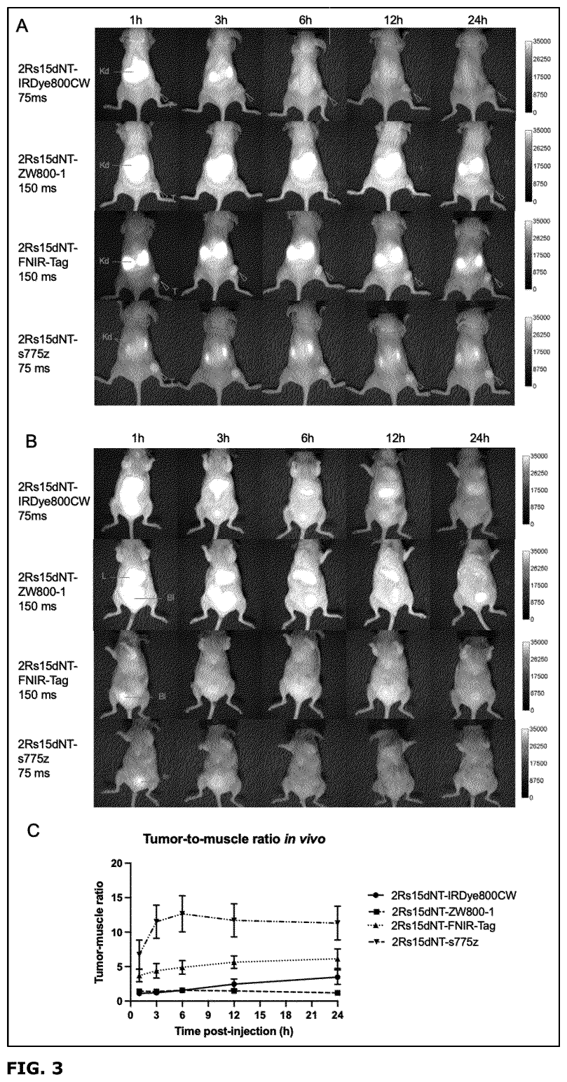

The whole body biodistribution and tumor targeting of the fluorescently-labeled anti-HER2 sdAbs was evaluated using non-invasive 2D fluorescence imaging (FIGS. 3A, B). Based on the acquired in vivo images, tumor-to-contralateral muscle ratios were subsequently quantified (FIG. 3C). For 2Rs15dNT-IRDye800CW, most intense fluorescent signals were observed in the kidneys, the bladder and the liver, although notable background signal was seen in all tissues the first hours post-injection (p.i.). As the background signal declined over time, the tumor became more clearly visible, with tumor-to-muscle ratios increasing from 1.10±0.31 at 1 h p.i. to 3.48±1.05 at 24 h p.i . . . . Similar biodistribution profile was obtained for 2Rs15dNT-ZW800-1,although tumor-to-muscle ratios remained low at all time points (1.41±0.23 at 1 h p.i. and 1.20±0.21 at 24 h p.i.). Interestingly, 2Rs15dNT-FNIR-Tag and in particular 2Rs15dNT-s775z (two conjugates according to the current invention) exhibited remarkably less overall background (besides the high renal and bladder signals). The tumor could therefore be clearly demarcated as soon as 1 h post-injection, though higher tumor-to-muscle ratios were obtained for 2Rs15dNT-s775z compared to 2Rs15dNT-FNIR-Tag (6.75±2.10 vs 3.70±0.90). These values further increase over 24 h, reaching 11.30±2.46 and 6.14±1.42 respectively.

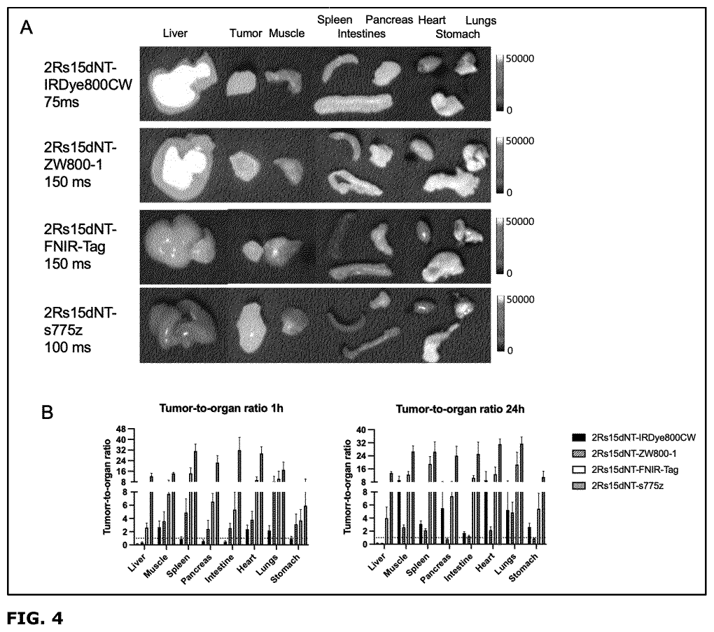

These observations were further confirmed by ex vivo analysis of the major organs and clearly indicated that highest tumor-to-organ ratios were obtained with 2Rs15dNT-FNIR-Tag and2Rs15dNT-s775z, two conjugates according to the current invention (FIGS. 4A, B). Importantly, only for those two tracers, the tumor is the most fluorescent tissue, even higher than the liver. This yielded tumor-to-liver ratios of respectively 2.61±0.68 and 12.29±2.22 as compared to 0.14±0.07 for 2Rs15dNT-IRDye800CW and 0.34±0.12 for 2Rs15dNT-ZW800-1. Other tumor-to-organ ratios for 2Rs15dNT-FNIR-Tag and2Rs15dNT-s775z at 1 and 24 h post-injection are consistently higher than for 2Rs15dNT-IRDye800CW and 2Rs15dNT-ZW800-1. Of note, the kidneys were exempted because they are considered as the major elimination route for sdAbs.

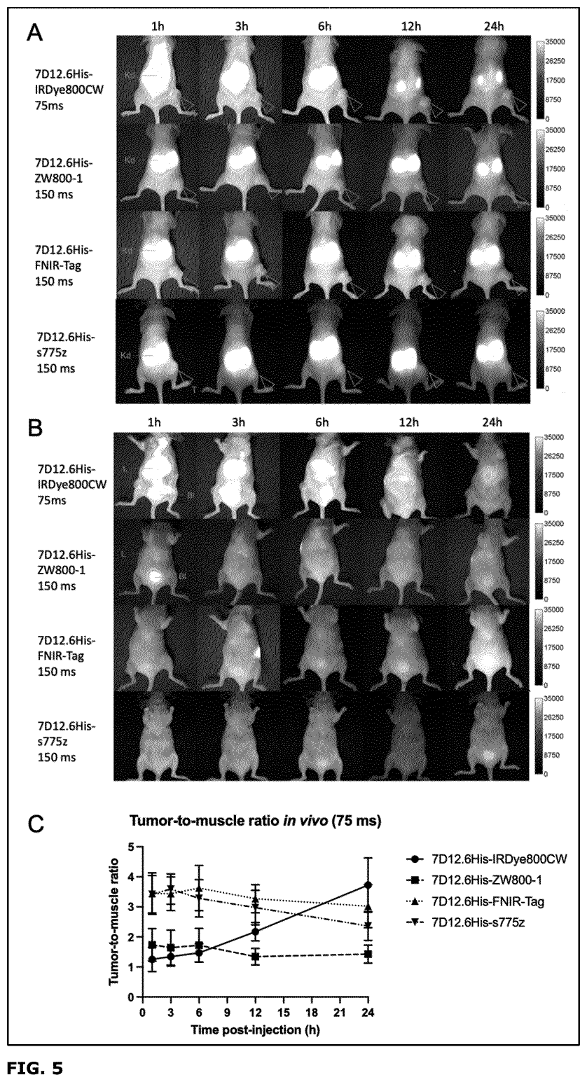

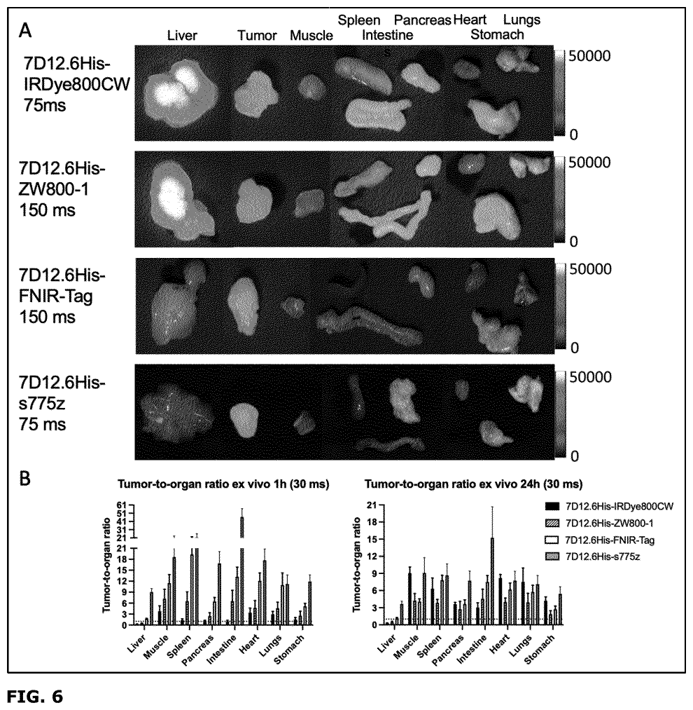

An analogous set of in vivo experiments was performed with the anti-EGFR sdAb 7D12.6His and similar results were obtained. FIGS. 5 and 6 show that both FNIR-and s775z-labeled 7D12.6His sdAbs exhibited a fast biodistribution profile with clearance via the kidneys, clear tumor uptake and very low background signals. This led to in vivo tumor-to-muscle ratios at 1 h post-injection of 3.45±0.69 for 7D12.6His-FNIR-Tag and 3.42±0.63 for 7D12.6His-s775z. The higher background seen with 7D12.6His-IRDye800CW and 7D12.6His-ZW800-1 resulted in tumor-to-muscle ratios at 1 h post-injection of only 1.25±0.41 and 1.73±0.54, respectively. These observations were further confirmed via ex vivo analysis (FIG. 6). In contrast to 7D12.6His-IRDye800CW and 7D12.6His-ZW800-1 for which the most fluorescent organ was the liver, the highest fluorescent signal for 7D12.6His-FNIR-Tag and 7D12.6His-s775z was seen in tumor tissue. This yielded respective tumor-to-liver ratios of 0.22±0.06, 0.41±0.15, 1.68±0.13 and 9.03±0.97 at 1 h. Ratios for all other resected organs/tissues were also consistently higher for 7D12.6His-FNIR-Tag and 7D12.6His-s775z as compared to 7D12.6His-IRDye800CW and 7D12.6His-ZW800-1 at early time points.

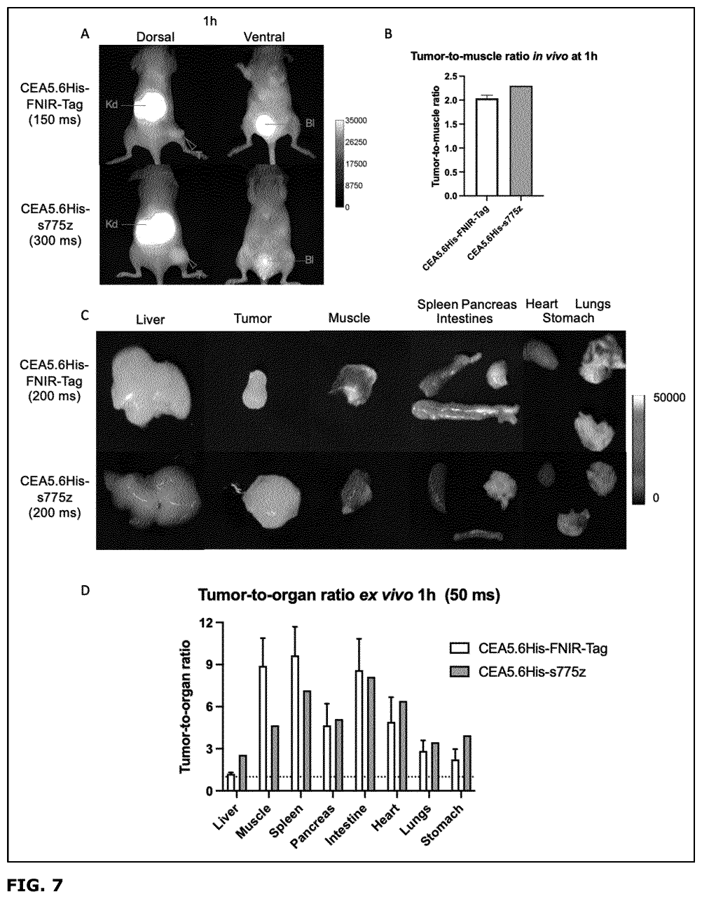

The results of the anti-CEA sdAb labeled with FNIR-Tag or s775z (FIG. 7) are in line with the other FNIR-Tag-and s775z-labeled sdAbs as described above. Except for the kidneys, highest fluorescent signal is observed in the tumor which allows clear delineation at early timepoints with in vivo tumor-to-contralateral muscle ratios above 2 at 1 h post-injection. Slightly higher liver signal is seen with CEA5.6His-FNIR-Tag as compared to CEA5.6His-s775z, but in both cases the tumor-to-liver ratio is above 1. Almost no background signal is seen in other resected tissues and organs.

### Conclusion

Surprisingly, FNIR-Tag and s775z-labeled sdAbs possess a clinically relevant biodistribution profile as compared to the IRDye800CW- or ZW800-1-labeled sdAbs. In particular, they exhibit fast blood clearance and low background signals in non-targeted organs, resulting in clear demarcation of tumor lesions at early time points after injection. FNIR-Tag or s775z-labeled sdAbs have a high potential for further implementation for clinical use.

These data further underline the importance of the chosen dye on the pharmacokinetic profile of a molecular tracer, and confirms that it is so far unpredictable what the impact will be for a specific class of molecular moiety.

As can be evidenced from FIGS. 3-6, conjugation of ZW800-1 to sdAbs does not improve the pharmacokinetics of the sdAbs as compared to IRDye800CW-conjugated sdAbs. Similar biodistribution profiles with high background values were obtained for both the ZW800-1 conjugates and the IRDye800CW conjugates. Furthermore, the tumor-to-background ratios remained also low at all time points, making it difficult to clearly discern the tumor.

This finding is in contrast to the results obtained for other targeting moieties, where pharmacokinetics of ZW800-conjugates show an improved pharmacokinetic profile compared to IRDye800CW-conjugates coupled to the same targeting moiety, the targeting moiety being chosen from a cyclic RGD peptide, fibrinogen or an antibody (Choi, H., Gibbs, S., Lee, J. et al. Targeted zwitterionic near-infrared fluorophores for improved optical imaging. Nat Biotechnol 31, 148-153 (2013). The article for instance describes that in tumor model systems, tumor-to-background ratios of 17.2 are achieved after only 4 h post-injection for ZW800-1, compared with 5.1 for IRDye800-CW. As such, one would expect that ZW800-sdAb conjugates would equally have an improved pharmacokinetic profile over IRDye800CW-sdAb conjugates. This is however not the case, as evidenced by the current application.

This clearly indicates that results regarding the favourable properties of a certain fluorophore coupled to a specific targeting moiety, can not be simply extrapolated to conjugates of said fluorophore coupled to a different (class of) targeting moiety.

### Example 2: Biodistribution And Tumor Targeting of an Anti-uPAR sdAb Conjugate According to the Current Invention

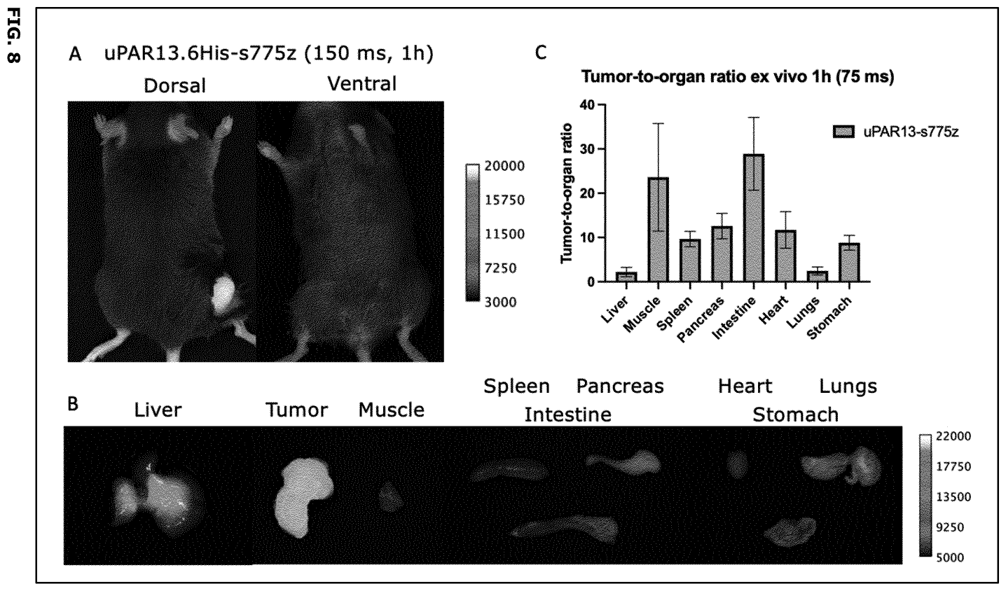

An anti-uPAR sdAb directed against mouse uPAR (uPAR13) coupled to a 6His tag (uPAR13.6His) was labeled with the near-infrared fluorescent dye s775z via amine-reactive conjugation chemistry. To this end, the sdAb was incubated for 2 h at pH 8.5 with a 5-fold molar excess of NHS-ester activated s775z dye and purified via size-exclusion chromatography. A degree of labeling of 1 on average was obtained and spectral characteristics of the dye remained close to those of the free dye (ex/em maxima: 780/802 nm). The s775z-labeled uPAR13.6His (a conjugate according to the current invention) was intravenously injected into MC38-tumor bearing mice (2 nmol based on dye concentration). In vivo dorsal and ventral fluorescence images acquired 1 h after injection of uPAR13.6His-s775z (FIG. 8A) and ex vivo images of dissected organs (FIG. 8B) show clear uptake of the conjugate according to the invention in the tumor, with low background signals and low liver uptake. Ex vivo tumor-to-organ ratios are displayed in FIG. 8C.

Examples 1 and 2 show data for 4 different sdAbs, all displaying favourable in vivo biodistribution and tumor targeting profiles when conjugated to a fluorescent moiety having a structure chosen from formula I or formula II, thereby forming conjugates of the invention. As such, we believe this favourable pharmacokinetic profile holds true for all sdAbs conjugated to a fluorescent moiety having a structure chosen from formula I or formula II.

Example 3: Comparison Between Biodistribution of R3B23.6His-s775z and R3B23.6His-IRDye800CW in Healthy Mice

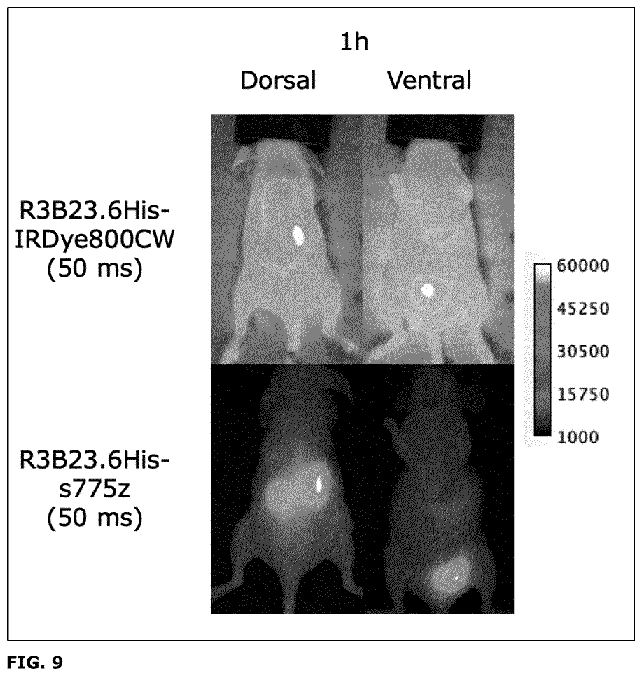

A sdAb which targets 5T2 multiple myeloma (MM) cell-produced M-protein, named R3B23.6His, was labeled with either s775z or IRDye800CW and was injected in healthy mice, functioning as a non-targeting control sdAb. Representative in vivo dorsal and ventral fluorescence images acquired 1 h after injection of R3B23.6His-IRDye800CW or R3B23.6His-s775z are displayed in FIG. 9. As in previous examples, R3B23.6His-IRDye800CW exhibits considerably higher background 5 signals over the whole body, as compared to the current invention (R3B23.6His-s775z).

The present invention is in no way limited to the embodiments described in the examples and/or shown in the figures. On the contrary, methods according to the present invention may be realized in many different ways without departing from the scope of the invention.

1. A conjugate comprising an immunoglobulin single variable domain conjugated to one or more detectable labels, wherein at least one of said labels comprises a fluorescent moiety, said fluorescent moiety having a structure chosen from formula I or formula II: 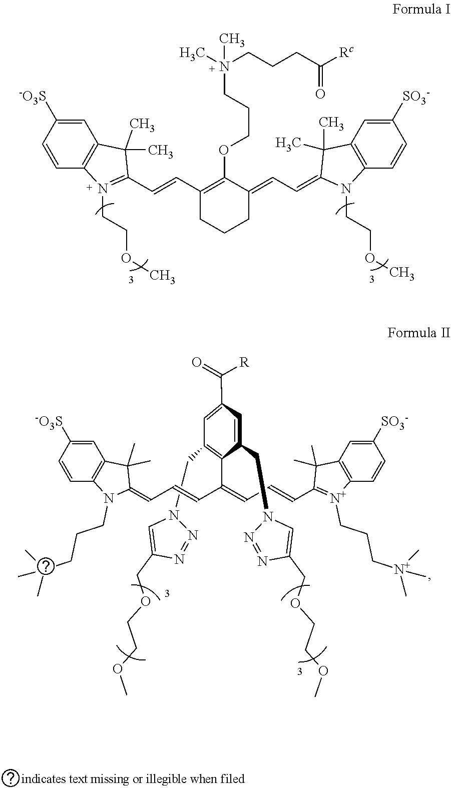
wherein R^{c} and R allow conjugation to the immunoglobulin single variable domain.

2. A conjugate comprising an immunoglobulin single variable domain conjugated to one or more detectable labels, characterized in that, said conjugate exhibits less than 10 percent serum protein binding as measured by HPLC.

3. The conjugate according to claim 2, wherein at least one of said labels comprises a fluorescent moiety, said fluorescent moiety having a structure chosen from formula I or formula II: 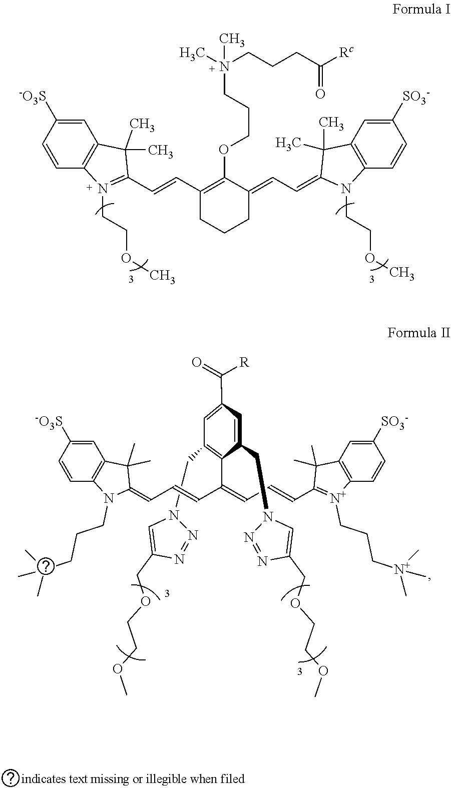
wherein R^{c} and R allow conjugation to the immunoglobulin single variable domain.

4. The conjugate according to claim 1, wherein said one or more detectable labels are conjugated to said immunoglobulin single variable domain by means of a functional group, wherein the functional group is an amine-reactive cross-linker or a sulfhydryl-reactive crosslinker.

5. The conjugate according to claim 1, wherein said one or more detectable labels comprise a bimodal label comprising a fluorescent moiety and a radionuclide.

6. The conjugate according to claim 5, wherein said radionuclide is chosen from the group of fluor 18 (¹⁸F), lutetium 177 (¹⁷⁷Lu), zirconium 89 (⁸⁹Zr), indium 111 (¹¹¹In), yttrium 90 (⁹⁰Y), copper 64 (⁶⁴Cu), gallium 67 (⁶⁷Ga), gallium 68 (⁶⁸Ga), technetium 99m (⁹⁹mTc), iodium 123 (¹²³I), iodium 124 (¹²⁴I), iodium 125 (¹²⁵I), or iodium 131 (¹³¹I).

7. The conjugate according to claim 1, wherein said immunoglobulin single variable domain is directed against and/or specifically binds to one or more clinically relevant targets in a body of a patient.

8. A pharmaceutical composition comprising the conjugate according to claim 1.

9. A method for in vivo medical imaging for prognostic, diagnostic and/or interventional purposes, wherein said method comprises administering the composition according to claim 8 to a patient.

10. The method for in vivo medical imaging according to claim 9, for diagnosis, prognosis and/or image-guided therapy of a disease or pathology.

11. The method for in vivo medical imaging according to claim 9 for diagnosis, prognosis and/or image-guided therapy of cancer, said cancer is selected from prostate cancer, liver cancer, head and/or neck cancer, brain cancer, skin cancer, bladder cancer, ovarian cancer, uterus cancer, lung cancer, breast cancer, sarcoma, cervix cancer, vulva cancer, hematologic cancers, renal cancer, bone cancer and/or gastrointestinal cancers.

12. The method for in vivo medical imaging according to claim 9, for endoscopic and/or intravascular imaging.

13. The method for in vivo medical imaging according to claim 9, wherein a dose of said conjugate is administered to a human patient, said dose ranging from 0.1 mg to 100 mg.

14. The conjugate according to claim 2, wherein said one or more detectable labels are conjugated to said immunoglobulin single variable domain by means of a functional group, wherein the functional group is an amine-reactive cross-linker or a sulfhydryl-reactive crosslinker.

15. The conjugate according to claim 2, wherein said one or more detectable labels comprise a bimodal label comprising a fluorescent moiety and a radionuclide.

16. The conjugate according to claim 15, wherein said radionuclide is chosen from the group of fluor 18 (¹⁸F), lutetium 177 (¹⁷⁷Lu), zirconium 89 (⁸⁹Zr), indium 111 (¹¹¹In), yttrium 90 (⁹⁰Y), copper 64 (⁶⁴Cu), gallium 67 (⁶⁷Ga), gallium 68 (⁶⁸Ga), technetium 99m (⁹⁹mTc), iodium 123 (¹²³I), iodium 124 (¹²⁴I), iodium 125 (¹²⁵I), or iodium 131 (¹³¹I).

17. The conjugate according to claim 2, wherein said immunoglobulin single variable domain is directed against and/or specifically binds to one or more clinically relevant targets in a body of a patient.

18. A pharmaceutical composition comprising the conjugate according to claim 2.

19. A method for in vivo medical imaging for prognostic, diagnostic and/or interventional purposes, wherein said method comprises administering the composition according to claim 18 to a patient.

20. The method for in vivo medical imaging according to claim 19, wherein a dose of said conjugate is administered to a human patient, said dose ranging from 0.1 mg to 100 mg.


SEQUENCE LISTING (5 sequences)

SEQ ID NO: 1 - protein, 124 aa, Vicugna pacos
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 2 - protein, 115 aa, Vicugna pacos
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 3 - protein, 123 aa, Vicugna pacos [sequence omitted]

SEQ ID NO: 4 - protein, 128 aa, Vicugna pacos
QVQLQESGGGSVQAGGSLRLSCAASGDTGYMGWFRQAPGKEREGVAVINSDSGVGSTYYADSVKGRFTISRDNAKNTVYLQMNSLKPEDTAIYYCAAGHFSDYVSPWTWREIYRYNVWGQGTQVTVSS

SEQ ID NO: 5 - protein, 121 aa, Vicugna pacos
QVQLQESGGGLVQSGGSLRLSCAVSGIGFVYYAMRWWRQAPGKERELVAGITGASGNTNYAESVKDRFTISRDNASNTVYLQMNSLKPEDTAVYYCNAAPLIRFGPPKDYWGQGTQVTVSS